(19) United States
(12) Patent Application Publication
(10) Pub. No.: US 2025/0061416 A1
(43) Pub. Date: Feb. 20, 2025

(54) CHEMICAL PRODUCT PASSPORT IN PRODUCT CHAINS

(71) Applicant: BASF SE, Ludwigshafen am Rhein (DE)

(72) Inventor: Henning SCHWABE, Ludwigshafen (DE)

(21) Appl. No.: 18/723,193

(22) PCT Filed: Dec. 19, 2022
(86) PCT No.: PCT/EP2022/086693

§ 371 Date: Jun. 21, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G06Q 10/087; G06Q 10/30

(52) U.S. Cl. CPC G06Q 10/087; G06Q 10/30; G06Q 2220/00

(57) ABSTRACT

Disclosed is an apparatus for generating a product passport, the apparatus comprising: one or more computing node(s) and one or more computer-readable media having thereon computer-executable instructions that are structured such that, when executed by the one or more computing node(s), cause the apparatus to perform the following steps:
receiving a request to provide a first decentral identifier associated with at least one first physical entity of a product supply chain and/or a recycling product chain;
in response to the request, providing the first decentral identifier associated with the at least one first physical entity of the product supply chain and/or the recycling product chain;
generating a product passport including the first decentral identifier and data related to the at least one first physical entity of the product supply chain and/or the recycling product chain;
providing the product passport for access by a data consuming service under control of a data providing service associated with a data owner.

### TECHNICAL FIELD

The present disclosure relates to an apparatus for generating a chemical product passport, a computer-implemented method for generating a product passport, a method for using a chemical product passport and a computer program element.

### TECHNICAL BACKGROUND

In the supply of chemical products multiple regulatory requirements need to be met, which differ depending on the chemical product. For instance, in automotive supply chains chemical companies provide standardized information using the International Chemical product Data System (IMDS). Such system allows to collect data along the entire automotive supply chain. Participants in the automotive supply chain register with the IMDS service and maintain product entries in the central database as provided and hosted by a third-party provider.

Systems like IMDS are static regarding data, prone to error and cumbersome in handling or maintenance. Owing to the highly specific and centralized setup of such systems, exchange and sharing of chemicals data is laborious. Hence there is a need to simplify chemical data exchange and sharing.

### SUMMARY OF THE INVENTION

In one aspect an apparatus for generating a product passport is disclosed, the apparatus comprising: one or more computing node(s) and one or more computer-readable media having thereon computer-executable instructions that are structured such that, when executed by the one or more computing node(s), cause the apparatus to perform the following steps:
receiving a request to provide a decentral identifier associated with at least one physical entity of a product supply chain, such as a chemical product supply chain, and/or a recycling product chain,
in response to the request, providing the decentral identifier associated with the at least one physical entity of the product supply chain, such as the chemical product supply chain, and/or a recycling product chain to a product passport generator for generating a product passport and/or to at least one authentication data registry.

The apparatus may comprise a product passport generator and/or at least one authentication data registry. The product passport generator may be configured to generate the product passport including the decentral identifier and data related to the at least one physical entity of the product supply chain, such as the chemical product supply chain, and/or a recycling product chain. The product passport generator may be configured to provide the product passport for access, e.g. to product data, by a data consuming service under control of or controlled by a data providing service associated with a data owner. The at least one authentication data registry may be configured to authenticate access, e.g. to product data, by a data consuming service.

In another aspect an apparatus for generating a product passport is disclosed, the apparatus comprising: one or more computing node(s) and one or more computer-readable media having thereon computer-executable instructions that are structured such that, when executed by the one or more computing node(s), cause the apparatus to perform the following steps:
receiving a request to provide a first decentral identifier associated with at least one first physical entity of a product supply chain, such as the chemical product supply chain, and/or a recycling product chain,
in response to the request, providing the first decentral identifier associated with the at least one first physical entity of the product supply chain, such as the chemical product supply chain, and/or the recycling product chain to a product passport generator for generating a product passport and/or to at least one authentication data registry.

The apparatus may comprise a product passport generator and/or at least one authentication data registry. The product passport generator may be configured to generate the product passport including the first decentral identifier and data related to the at least one first physical entity of the product supply chain, such as the chemical product supply chain, and/or a recycling product chain. The product passport generator may be configured to provide the product passport for access, e.g. to product data, by a data consuming service under control of or controlled by a data providing service associated with a data owner. The at least one authentication data registry may be configured to authenticate access, e.g. to product data, by a data consuming service.

In another aspect an apparatus for generating a product passport is disclosed, the apparatus comprising: one or more computing node(s), and one or more computer-readable media having thereon computer-executable instructions that are structured such that, when executed by the one or more computing node(s), cause the apparatus to perform the following steps:
receiving a request to provide a first decentral identifier associated with at least one first physical entity of a product supply chain, such as a chemical product supply chain, and/or a recycling product chain;
in response to the request, providing the first decentral identifier associated with the at least one first physical entity of the product supply chain, such as the chemical product supply chain, and/or a recycling product chain;
providing at least one second decentral identifier, wherein the at least one second decentral identifier is associated with at least one second physical entity of the chemical product supply chain, such as the chemical product supply chain, and/or the recycling product chain;
generating a concatenation based on a relationship representation according to which the first decentral identifier is associated with the at least one second decentral identifier;
providing the first decentral identifier, the at least one second decentral identifier and/or the concatenation to a product passport generator for generating a product passport and/or to at least one authentication data registry.

The apparatus may comprise a product passport generator and/or at least one authentication data registry. The product passport generator may be configured to generate the product passport including
the first decentral identifier,
data related to the at least one first physical entity of the product supply chain, such as the chemical product supply chain, and/or the recycling product chain,
the second decentral identifier and/or
the concatenation,

The product passport generator may be configured to provide the product passport for access, e.g. to product data, by a data consuming service under control of or controlled by a data providing service associated with a data owner. The at least one authentication data registry may be configured to authenticate access by a data consuming service and/or to resolve the concatenation.

In another aspect an apparatus for generating a product passport is disclosed, the apparatus comprising: one or more computing nodes, and one or more computer-readable media having thereon computer-executable instructions that are structured such that, when executed by the one or more computing nodes, cause the apparatus to perform the following steps:
providing a first decentral identifier associated with at least one first physical entity of a product supply chain, such as a chemical product supply chain, and/or a recycling product chain;
providing at least one second decentral identifier, wherein the at least one second decentral identifier is associated with at least one second physical entity of the product supply chain, such as the chemical product supply chain, and/or the recycling product chain;
generating a concatenation based on a relationship representation according to which the first decentral identifier is associated with the second decentral identifier;
providing the first decentral identifier, the at least one second decentral identifier and/or the concatenation to a product passport generator for generating a product passport and/or to at least one authentication data registry.

The apparatus may comprise a product passport generator and/or at least one authentication data registry. The product passport generator may be configured to generate the product passport including
the first decentral identifier,
data related to the at least one first physical entity of the product supply chain, such as the chemical product supply chain, and/or the recycling product chain,
the second decentral identifier and/or
the concatenation,

The product passport generator may be configured to provide the product passport for access, e.g. to product data, by a data consuming service under control of or controlled by a data providing service associated with a data owner. The at least one authentication data registry may be configured to authenticate access by a data consuming service and/or to resolve the concatenation.

In another aspect an apparatus for generating a product passport is disclosed, the apparatus comprising: one or more computing nodes; and one or more computer-readable media having thereon computer-executable instructions that are structured such that, when executed by the one or more computing nodes, cause the apparatus to perform the following steps:
receiving a request to provide a decentral identifier associated with at least one physical entity of the product supply chain, such as the chemical product supply chain, and/or the recycling product chain;
in response to the request, providing the decentral identifier associated with the at least one physical entity of the product supply chain, such as the chemical product supply chain, and/or the recycling product chain;
generating a product passport including the decentral identifier and data related to the at least one physical entity of the product supply chain, such as the chemical product supply chain, and/or the recycling product chain;
providing the product passport for access, e.g. to product data, by a data consuming service under control of or controlled by a data providing service associated with a data owner.

In one aspect an apparatus for generating a product passport is disclosed, the apparatus comprising: one or more computing nodes; and one or more computer-readable media having thereon computer-executable instructions that are structured such that, when executed by the one or more computing nodes, cause the apparatus to perform the following steps:
receiving a request to provide a first decentral identifier associated with at least one first physical entity of the product supply chain, such as the chemical product supply chain, and/or the recycling product chain;
in response to the request, providing the first decentral identifier associated with the at least one first physical entity of the product supply chain, such as the chemical product supply chain, and/or the recycling product chain;
generating a product passport including the first decentral identifier and data related to the at least one first physical entity of the product supply chain, such as the chemical product supply chain, and/or the recycling product chain;
providing the product passport for access, e.g. to product data, by a data consuming service under control of or controlled by a data providing service associated with a data owner.

In another aspect an apparatus for generating a product passport is disclosed, the apparatus comprising: one or more computing nodes; and one or more computer-readable media having thereon computer-executable instructions that are structured such that, when executed by the one or more computing nodes, cause the apparatus to perform the following steps:
receiving a request to provide a first decentral identifier associated with at least one first physical entity of the product supply chain, such as the chemical product supply chain, and/or the recycling product chain;
in response to the request, providing the first decentral identifier associated with the at least one first physical entity of the product supply chain, such as the chemical product supply chain, and/or the recycling product chain;
providing at least one second decentral identifier, wherein the at least one second decentral identifier is associated with at least one second physical entity of the product supply chain, such as the chemical product supply chain, and/or the recycling product chain,
generating a concatenation based on a relationship representation according to which the first decentral identifier is associated with the at least one second decentral identifier;
generating a product passport including the first decentral identifier, the at least one second decentral identifier and/or the concatenation;
providing the product passport for access, e.g. to product data, by a data consuming service under control of or controlled by a data providing service associated with a data owner.

In another aspect an apparatus for generating a product passport is disclosed, the apparatus comprising: one or more computing nodes; and one or more computer-readable media having thereon computer-executable instructions that are structured such that, when executed by the one or more computing nodes, cause the apparatus to perform the following steps:
providing a first decentral identifier associated with at least one first physical entity of a product supply chain, such as the chemical product supply chain, and/or a recycling product chain;
providing at least one second decentral identifier, wherein the at least one second decentral identifier is associated with at least one second physical entity of the product supply chain, such as the chemical product supply chain, and/or the recycling product chain,
generating a concatenation based on a relationship representation according to which the first decentral identifier is associated with the at least one second decentral identifier;
generating a product passport including the first decentral identifier, the at least one second decentral identifier and/or the concatenation;
providing the product passport for access, e.g. to product data, by a data consuming service under control of or controlled by a data providing service associated with a data owner.

In another aspect a computer-implemented method for generating a product passport is disclosed, the method comprising the steps:
receiving a request to provide a decentral identifier associated with at least one physical entity of a product supply chain, such as the chemical product supply chain, and/or a recycling product chain,
in response to the request, providing the decentral identifier associated with the at least one physical entity of the chemical product supply chain, such as the chemical product supply chain, and/or the recycling product chain,
providing the decentral identifier to a product passport generator for generating a product passport and/or to at least one authentication data registry.

In another aspect a computer-implemented method for generating a product passport is disclosed, the method comprising the steps:
receiving a request to provide a first decentral identifier associated with at least one first physical entity of a product supply chain, such as the chemical product supply chain, and/or a recycling product chain,
in response to the request, providing the first decentral identifier associated with the at least one first physical entity of the product supply chain, such as the chemical product supply chain, and/or the recycling product chain,
providing the first decentral identifier to a product passport generator for generating a product passport and/or to at least one authentication data registry.

The method may comprise product passport generation and/or authentication. The method may comprise generating the product passport including the first decentral identifier and data related to the at least one first physical entity of the product supply chain, such as the chemical product supply chain, and/or the recycling product chain. The method may comprise providing the product passport for access, e.g. to product data, by a data consuming service under control of or controlled by a data providing service associated with a data owner. The method may comprise authenticating access, e.g. to product data, by a data consuming service via at least one authentication data registry.

In another aspect a computer-implemented method for generating a product passport is disclosed, the method comprising the steps:
receiving a request to provide a first decentral identifier associated with at least one first physical entity of a product supply chain, such as the chemical product supply chain, and/or a recycling product chain,
in response to the request, providing the first decentral identifier associated with the at least one first physical entity of the product supply chain, such as the chemical product supply chain, and/or a recycling product chain;
providing at least one second decentral identifier, wherein the at least one second decentral identifier is associated with at least one second physical entity of the product supply chain, such as the chemical product supply chain, and/or the recycling product chain,
generating a concatenation based on a relationship representation according to which the first decentral identifier is associated with the at least one second decentral identifier;
providing the first decentral identifier, the at least one second decentral identifier and/or the concatenation to a product passport generator for generating a product passport and/or to at least one authentication data registry.

The method may comprise product passport generation and/or authentication. The method may comprise generating the product passport including
the first decentral identifier,
data related to the at least one first physical entity of the product supply chain, such as the chemical product supply chain, and/or the recycling product chain,
the second decentral identifier and/or
the concatenation.

The method may comprise providing the product passport for access, e.g. to product data, by a data consuming service under control of or controlled by a data providing service associated with a data owner. The method may comprise authenticating access, e.g. to product data, by a data consuming service and/or resolving the concatenation at least one authentication data registry.

In another aspect a computer-implemented method for generating a product passport is disclosed, the method comprising the steps:
providing a first decentral identifier associated with at least one first physical entity of a product supply chain, such as the chemical product supply chain, and/or a recycling product chain;
providing at least one second decentral identifier, wherein the at least one second decentral identifier is associated with at least one second physical entity of the product supply chain, such as the chemical product supply chain, and/or the recycling product chain;
generating a concatenation based on a relationship representation according to which the first decentral identifier is associated with the second decentral identifier;
providing the first decentral identifier, the at least one second decentral identifier and/or the concatenation to a product passport generator for generating a product passport and/or to at least one authentication data registry.

The method may comprise product passport generation and/or authentication. The method may comprise generating the product passport including
the first decentral identifier,
data related to the at least one first physical entity of the product supply chain, such as the chemical product supply chain, and/or the recycling product chain,
the second decentral identifier and/or
the concatenation.

The method may comprise providing the product passport for access, e.g. to product data, by a data consuming service under control of or controlled by a data providing service associated with a data owner. The method may comprise authenticating access, e.g. to product data, by a data consuming service and/or resolving the concatenation at least one authentication data registry.

In another aspect a computer-implemented method for generating a product passport is disclosed, the method comprising the steps:
receiving a request to provide a decentral identifier associated with one physical entity of the product supply chain, such as the chemical product supply chain, and/or a recycling product chain;
in response to the request, providing the decentral identifier associated with the at least one physical entity of the product supply chain, such as the chemical product supply chain, and/or a recycling product chain;
generating a product passport including the decentral identifier and data related to the at least one physical entity of the product supply chain, such as the chemical product supply chain, and/or a recycling product chain;
providing the product passport for access, e.g. to product data, by a data consuming service under control of or controlled by a data providing service associated with a data owner.

In another aspect a computer-implemented method for generating a product passport is disclosed, the method comprising the steps:
receiving a request to provide a first decentral identifier associated with one first physical entity of the product supply chain, such as the chemical product supply chain, and/or a recycling product chain;
in response to the request, providing the first decentral identifier associated with the at least one first physical entity of the product supply chain, such as the chemical product supply chain, and/or a recycling product chain;
generating a product passport including the first decentral identifier and data related to the at least one first physical entity of the product supply chain, such as the chemical product supply chain, and/or a recycling product chain;
providing the product passport for access, e.g. to product data, by a data consuming service under control of or controlled by a data providing service associated with a data owner.

In another aspect a computer-implemented method for generating a product passport is disclosed, the method comprising the steps:
receiving a request to provide a first decentral identifier associated with at least one first physical entity of a product supply chain-, such as the chemical product supply chain, and/or a recycling product chain;
in response to the request, providing the first decentral identifier associated with the at least one first physical entity of the product supply chain, such as the chemical product supply chain, and/or the recycling product chain;
providing at least one second decentral identifier, wherein the at least one second decentral identifier is associated with at least one second physical entity of the product supply chain, such as the chemical product supply chain, and/or the recycling product chain,
generating a concatenation based on a relationship representation according to which the first decentral identifier is associated with the at least one second decentral identifier;
generating a product passport including the first decentral identifier, the at least one second decentral identifier and/or the concatenation;
providing the product passport for access, e.g. to product data, by a data consuming service under control of or controlled by a data providing service associated with a data owner.

In another aspect a computer-implemented method for generating a product passport is disclosed, the method comprising the steps:
providing a first decentral identifier associated with at least one first physical entity of a product supply chain, such as the chemical product supply chain, and/or a recycling product chain;
providing at least one second decentral identifier, wherein the at least one second decentral identifier is associated with at least one second physical entity of the product supply chain, such as the chemical product supply chain, and/or the recycling product chain,
generating a concatenation based on a relationship representation according to which the first decentral identifier is associated with the at least one second decentral identifier;
generating a product passport including the first decentral identifier, the at least one second decentral identifier and/or the concatenation;
providing the product passport for access, e.g. to product data, by a data consuming service under control of or controlled by a data providing service associated with a data owner.

### Further Aspects may Relate to Producing, Providing or Recycling a Product

In another aspect the disclosure relates to an apparatus for producing, providing or recycling a product associated with a product passport, wherein the product comprises at least one first physical entity of a product supply chain and/or a recycling product chain, the apparatus comprising:
at least one collector configured to collect product data associated with at least one first physical entity of the product supply chain and/or the recycling product chain, wherein the at least one first physical entity comprises at least one physical identifier;
at least one assignor configured to assign the physical identifier to a first decentral identifier for generating the product passport associated with the at least one first physical entity;
a product passport generator configured to generate the product passport by receiving a request to provide at least the first decentral identifier associated with product data of the at least one first physical entity and in response to the request, generating the product passport including the first decentral identifier and data related to the product data of the at least one first physical entity.

In another aspect the disclosure relates to a computer-implemented method for producing, providing or recycling a product associated with a product passport, wherein the product comprises at least one first physical entity of a product supply chain and/or a recycling product chain, the method comprising the steps:
collecting product data associated with at least one first physical entity of the product supply chain and/or the recycling product chain, wherein the at least one first physical entity comprises at least one physical identifier;
assigning the physical identifier to a first decentral identifier for generating the product passport associated with the at least one first physical entity;
generating the product passport by receiving a request to provide at least the first decentral identifier associated with product data of the at least one first physical entity and in response to the request, generating the product passport including the first decentral identifier and data related to the product data of the at least one first physical entity.

In another aspect the disclosure relates to a computer-implemented method for producing, providing or recycling a product associated with a product passport, wherein the product comprises at least one first physical entity of a product supply chain and/or a recycling product chain, the method comprising the steps:
producing the first physical entity connected to or comprising a physical identifier;
optionally collecting product data associated with at least one first physical entity of the product supply chain and/or the recycling product chain, wherein the at least one first physical entity comprises at least one physical identifier;
assigning the physical identifier to a first decentral identifier for generating the product passport associated with the at least one first physical entity;
generating the product passport by receiving a request to provide at least the first decentral identifier associated with product data of the at least one first physical entity and in response to the request, generating the product passport including the first decentral identifier and data related to the product data of the at least one first physical entity.

In another aspect the disclosure relates to a system for producing, providing or recycling a product associated with a product passport, wherein the product comprises at least one first physical entity of a product supply chain and/or a recycling product chain, the apparatus comprising:
a production line configured to produce the product and/or a recycling line configured to recycle the product connected to or comprising a physical identifier;
optionally at least one collector configured to collect product data associated with at least one first physical entity of the product supply chain and/or the recycling product chain, wherein the at least one first physical entity comprises at least one physical identifier;
at least one assignor configured to assign the physical identifier to a first decentral identifier for generating the product passport associated with the at least one first physical entity, such as a produced product and/or recycled product;
a product passport generator configured to generate the product passport by receiving a request to provide at least the first decentral identifier associated with product data of the at least one first physical entity and in response to the request, generating the product passport including the first decentral identifier and data related to the product data of the at least one first physical entity.

In yet another aspect a computer-implemented method for using a product passport, preferably to further process the product, preferably the chemical product, associated with the product passport, preferably the chemical product passport, is disclosed, the method comprising the steps:
receiving a request to access and/or process the product passport associated with a first decentral identifier as generated according to the methods or by the apparatuses lined out herein;
optionally authenticating the product passport and/or the request to access and/or process the product passport;
based on the product passport and optionally the authentication, providing access to product data associated with at least one decentral identifier, preferably the first decentral identifier or at least one second decentral identifier, of the product passport.

In yet another aspect a product associated with the product passport is disclosed, wherein the product passport including the decentral identifier, e.g. the first decentral identifier, and data related to the product data is generated for the product according to the methods or by the apparatus lined out herein.

In yet another aspect a system including a product associated with the product passport is disclosed, wherein the product passport includes a decentral identifier, e.g. the first decentral identifier, and data related to the product data and is generated for the product according to the methods or by the apparatuses lined out herein.

In yet another aspect a product passport including a decentral identifier, e.g. the first decentral identifier, and data related to the product data is disclosed, wherein the product passport is generated for the product according to the methods or by the apparatuses lined out herein.

In yet another aspect a computer element, in particular a computer program product or a computer readable medium, with instructions, which when executed on one or more computing node(s) is configured to carry out the steps of any of the methods disclosed herein or carry out the steps performed by the apparatuses disclosed herein.

### Further Aspects may Include

In one aspect, an apparatus for producing, providing or recycling a product, preferably a chemical product, associated with a product passport is disclosed, the apparatus comprising:
at least one collector configured to collect product data associated with the product with one or more physical entities of the product of a product supply chain and/or with one or more physical entities of the product of a recycling product chain, wherein the product comprises at least one physical identifier;
at least one assignor configured to assign the physical identifier to a decentral identifier for generating the product passport associated with the produced product or recycled product;
a product passport generator configured to generate the product passport by receiving a request to provide at least the decentral identifier associated with product data of the product in the product supply chain and/or the recycling product chain and in response to the request, generating the product passport including the decentral identifier and data related to the product data of the product in the product supply chain and/or the recycling product chain.

In another aspect an apparatus for generating a product passport is disclosed, the apparatus comprising: one or more computing nodes; and one or more computer-readable media having thereon computer-executable instructions that are structured such that, when executed by the one or more computing nodes, cause the apparatus to perform the following steps:
receiving a request to provide a decentral identifier associated with at least one physical entity of product supply chain and/or the recycling product chain,
in response to the request, providing a decentral identifier associated with the at least one physical entity of product supply chain and/or the recycling product chain,
providing a decentral identifier to a product passport generator for generating a product passport and to at least one authentication data registry.

In another aspect, an apparatus for generating a product passport is disclosed, the apparatus comprising: one or more computing nodes; and one or more computer-readable media having thereon computer-executable instructions that are structured such that, when executed by the one or more computing nodes, cause the apparatus to perform the following steps:
receiving a request to provide a decentral identifier associated with product data of the product in the product supply chain and/or the recycling product chain;
in response to the request, generating the product passport including the decentral identifier and data related to the product in the product supply chain and/or the recycling product chain.

In another aspect, a computer-implemented method for generating a product passport is disclosed, the method comprising the steps:
receiving a request to provide a decentral identifier associated with product data of the product in the product supply chain and/or the recycling product chain and preferably a data owner;
in response to the request, generating the product passport including the decentral identifier and data related to the product data of the product in the product supply chain and/or the recycling product chain;
and preferably, providing the product passport for access by a data consuming service controlled by a data providing service associated with a data owner.

In a further aspect of the present disclosure, an apparatus for generating a product passport is disclosed, the apparatus comprising:
at least one receiving unit configured to receive a request to provide a decentral identifier associated with product data of the product in the product supply chain and/or the recycling product chain;
at least one generating unit configured to generate the product passport, in response to the request, including the decentral identifier and data related to the product data of the product in the product supply chain and/or the recycling product chain.

A further aspect of the present disclosure relates to a method for producing, providing or recycling a product, e.g. a chemical product, associated with a product passport, the method comprising:
producing the product connected to or comprising a physical identifier;
assigning the physical identifier to a decentral identifier for generating the product passport associated with the produced product;
generating the product passport according to the computer-implemented method for generating a product passport or by the system or apparatus for generating a product passport.

A further aspect of the present disclosure relates to a system for producing, providing or recycling a product and/or a recycled product associated with a product passport, the system comprising:
a production line configured to produce the product and/or a recycling line configured to recycle the product connected to or comprising a physical identifier;
an assignor configured to assign the physical identifier to a decentral identifier for generating the product passport associated with the produced product and/or recycled product;
a product passport generator configured to generate the product passport by receiving a request to provide a decentral identifier associated with product data of the product in the product supply chain and/or the recycling product chain;
and in response to the request, generating the product passport including the decentral identifier and data related to the product.

A further aspect of the present disclosure relates to a computer-Implemented method for producing, providing or recycling a product associated with a product passport, the method comprising:
based on a physical identifier of the product, requesting to provide a decentral identifier and assigning the decentral identifier to the physical identifier;
in response to the request, the product passport including the decentral identifier and data related to the produced and/or recycled product is generated according to the computer-implemented method for generating a product passport or by the system or apparatus for generating a product passport.

In a further aspect, a computer-implemented method for producing, providing or recycling associated with a product passport is disclosed, the method comprising:
collecting product data associated with the product with one or more physical entities of the product of a product supply chain and/or with one or more physical entities of the product of a recycling product chain, wherein the product comprises at least one physical identifier;
assigning the physical identifier to a decentral identifier for generating the product passport associated with the produced product or recycled product;
generating the product passport by receiving a request to provide at least the decentral identifier associated with product data of the product in the product supply chain and/or the recycling product chain and in response to the request, generating the product passport including the decentral identifier and data related to the product data of the product in the product supply chain and/or the recycling product chain.

In a further aspect an apparatus for generating a product passport is disclosed, the apparatus comprising: one or more computing nodes; and one or more computer-readable media having thereon computer-executable instructions that are structured such that, when executed by the one or more computing nodes, cause the apparatus to perform the following steps:
receiving a request to provide a decentral identifier associated with one physical entity of product supply chain and/or the recycling product chain
in response to the request, providing a decentral identifier associated with the at least one physical entity of product supply chain and/or the recycling product chain
generating a product passport including the decentral identifier and data related to the at least one physical entity of product supply chain and/or the recycling product chain;
providing the product passport for access by a data consuming service controlled by a data providing service associated with a data owner.

In another aspect a computer-implemented method for generating a product passport is disclosed, the method comprising the steps:
receiving a request to provide a decentral identifier associated with at least one physical entity of product supply chain and/or the recycling product chain,
in response to the request, providing a decentral identifier associated with the at least one physical entity of product supply chain and/or the recycling product chain,
providing a decentral identifier to a product passport generator for generating a product passport and to at least one authentication data registry.

In another aspect a computer-implemented method for generating a product passport is disclosed, the method comprising the steps:
receiving a request to provide a decentral identifier associated with one physical entity of product supply chain and/or the recycling product chain
in response to the request, providing a decentral identifier associated with the at least one physical entity of product supply chain and/or the recycling product chain
generating a product passport including the decentral identifier and data related to the at least one physical entity of product supply chain and/or the recycling product chain;
providing the product passport for access by a data consuming service controlled by a data providing service associated with a data owner.

In yet another aspect a computer-implemented method for using a product passport, preferably to further process the product associated with the product passport, is disclosed, the method comprising the steps:
receiving a request to access and/or process the product passport associated with a decentral identifier as generated according to the methods or by the apparatuses lined out herein;
optionally authenticating the product passport and/or the request to access and/or process the product passport;
based on the product passport and optionally the authentication, providing access to product data associated with at least one decentral identifier of the product passport.

A further aspect of the present disclosure relates to an apparatus for producing, providing or recycling a product associated with a product passport, the apparatus comprising:
one or more computing nodes and one or more computer-readable media having thereon computer-executable instructions that are structured such that, when executed by the one or more computing nodes, cause the apparatus to perform the following steps:
based on a physical identifier of the product, requesting to provide a decentral identifier and assigning the decentral identifier to the physical identifier;
in response to the request, generating the product passport including the decentral identifier and data related to the produced and/or recycled product, wherein the product passport is generated by receiving a request to provide a decentral identifier associated with product data of the product in the product supply chain and/or the recycling product chain and in response to the request, generating the product passport including the decentral identifier and data related to the product in the product supply chain and/or the recycling product chain.

A further aspect of the present disclosure relates to an apparatus for producing, providing or recycling a product associated with a product passport, the apparatus comprising:
a requestor configured to request, based on a physical identifier of the product, to provide a decentral identifier;
an assignor configured to assign the decentral identifier to the physical identifier;
a product passport generator configured to generate, in response to the request, the product passport including the decentral identifier and data related to the produced and/or recycled product, wherein the product passport is generated by receiving a request to provide a decentral identifier associated with data of the product in the product supply chain and/or the recycling product chain and in response to the request, generating the product passport including the decentral identifier and data related to the product in the product supply chain and/or the recycling product chain.

In a further aspect an apparatus for producing, providing or recycling a product associated with a product passport is disclosed, the apparatus comprising:
a collector configured to collect product data associated with the product with one or more physical entities of the product of a product supply chain and/or with one or more physical entities of the product of a recycling product chain, wherein the product is connected to or comprises the physical identifier;
an assignor configured to assign the physical identifier to a decentral identifier for generating the product passport associated with the produced and/or recycled product;
a product passport generator configured to generate the product passport by receiving a request to provide a decentral identifier associated with product data of the product and in response to the request, generating the product passport including the decentral identifier and data related to product data of the produced and/or recycled product.

In a further aspect, a computer-implemented method for producing, providing or recycling a product associated with a product passport is disclosed, the method comprising:
collecting product data associated with the product with one or more physical entities of the product of a product supply chain and/or with one or more physical entities of the product of a recycling product chain, wherein the product is connected to or comprises the physical identifier;
generating the product passport includes receiving a request to provide a decentral identifier associated with product data of the product and in response to the request, generating the product passport including the decentral identifier and data related to product data of the product.

In a further aspect a system for producing, providing or recycling a product associated with a product passport is disclosed, the system comprising:
a production line configured to produce the product and/or a recycling line configured to recycle the product connected to or comprising a physical identifier;
a collector configured to collect product data associated with the product data associated with the production and/or recycling of the product, wherein the product is connected to or comprises the physical identifier;
an assignor configured to assign the physical identifier to a decentral identifier for generating the product passport associated with the produced product and/or recycled product;
a product passport generator configured to generate the product passport by receiving a request to provide a decentral identifier associated with product data of the product and in response to the request, generating the product passport including the decentral identifier and data related to product data of the product.

In yet another aspect, a computer-implemented method for using a product passport, preferably to further process the product associated with the product passport, is disclosed, the method comprising the steps:
receiving a request to access the product data associated with a decentral identifier of the product passport as generated according to the methods disclosed herein or by the apparatuses disclosed herein;
optionally authenticating and/or authorizing the request to access the product data;
based on optionally the authentication and/or authorization, providing access to product data associated with the decentral identifier of the product passport.

In a further aspect, a system for producing, providing or recycling a product associated with a product passport is disclosed, the system comprising:
a production line configured to produce the product and/or a recycling line configured to recycle the product connected to or comprising a physical identifier;
a collector configured to collect product data associated with the product or product data associated with the production and/or recycling of the product, wherein the product is connected to or comprises the physical identifier;
an assignor configured to assign the physical identifier to a decentral identifier for generating the product passport associated with the produced and/or recycled product;
a product passport generator configured to generate the product passport by receiving a request to provide a decentral identifier associated with product data of the product and in response to the request, generating the product passport including the decentral identifier and data related to product data of the product.

In a further aspect, a method for producing, providing or recycling a product associated with a product passport is disclosed, the method comprising:
producing and/or recycling the product connected to or comprising a physical identifier;
collecting product data associated with the product or product data associated with the production and/or recycling of the product, wherein the product is connected to or comprises the physical identifier;
assigning the physical identifier to the decentral identifier for generating the product passport associated with the produced and/or recycled product, wherein generating the product passport includes receiving a request to provide a decentral identifier associated with product data of at least the product and in response to the request, generating the product passport including the decentral identifier and data related to the product data of the product.

A further aspect of the present disclosure relates to a use of the product passport as generated according to the computer-implemented method for generating a product passport or by the system or apparatus for generating a product passport to further process and/or recycle the product and/or the product associated with the product passport.

A further aspect of the present disclosure relates to a use of the product passport as generated according to the computer-implemented method for producing, providing or recycling a product associated with a product passport or by the system or apparatus for producing, providing or recycling a product associated with a product passport to further process the product and/or the product associated with the product passport.

A further aspect of the present disclosure relates to a product and/or a product associated with the product passport, wherein the product passport including one or more decentral identifiers and data related to the product data is generated according to the computer-implemented method for generating a product passport or by the system or apparatus for generating a product passport.

A further aspect of the present disclosure relates to a product and/or a product associated with the product passport, wherein the product passport including one or more decentral identifiers and data related to the product data is generated according to the computer-implemented method for producing, providing or recycling a product associated with a product passport or by the system or apparatus for producing or providing a product associated with a product passport.

A further aspect of the present disclosure relates to a product passport including one or more decentral identifier(s) and data related to the product data, wherein the product passport is generated according to the computer-implemented method for generating a product passport or by the system or apparatus for generating a product passport.

A further aspect of the present disclosure relates to a product passport including one or more decentral identifier(s) and data related to the product data, wherein the product passport is generated according to the computer-implemented method for producing, providing or recycling a product associated with a product passport or by the system or apparatus for producing, providing or recycling a product associated with a product passport.

A further aspect of the present disclosure relates to a computer program element with instructions, which when executed on one or more computing node(s) is configured to carry out the steps of the computer-implemented method for generating a product passport or for producing, providing or recycling a product associated with a product passport.

Any disclosure and embodiments described herein relate to the methods, the apparatuses, the systems, the products, the product passports, the computer elements lined out above or below. The benefits provided by any of the embodiments and examples equally apply to all other embodiments and examples.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

The methods, the apparatuses, the systems, the products, the product passports and the computer elements disclosed herein provide an efficient, secure and robust way for sharing or exchanging data across different participant nodes in chemical value chains. In particular, a) by attaching a decentral identifier to a data owner and associated chemical product data and b) by providing access by a data consuming service controlled by a data providing service associated with the data owner, data can be securely exchanged and shared under the sovereignty of the data owner. The data owner may thus control access by participant nodes or data consuming services of the decentral network to the product data. This allows for simplified and customizable data sharing or exchange from chemical industry to chemical supply chain participants. This way, a more reliable and efficient further processing of supplied chemical product by upstream participants of the chemical supply chain can be achieved, while the data remains in the ownership of the chemical supplier supplying the upstream participant. By combining the data related to chemicals directly with the decentral identifier and optionally one or more authentication mechanisms more reliable and secure data sharing and exchange can be provided. By further including one or more authorization mechanisms, the data sharing or exchange can be conducted in a more flexible manner with multiple data consuming services from different participants of the chemical supply chain accessing the chemical product data. By concatenating decentral identifiers associated with products of the product supply chain, such as the chemical product supply chain, and/or the recycling product chain, the physical entities can be tracked and traced virtually. The physical entities at any stage of the product supply chain, such as the chemical product supply chain, and/or the recycling product chain may be tracked and traced through concatenation.

The term “decentral identifier” is to be understood broadly in the present case. The decentral identifier may comprise any unique identifier uniquely associated with the data owner and product data, such as chemical product data. The decentral identifier may include one or more Universally Unique Identifier(s) (UUID) or a Digital Identifier(s) (DID) or decentralized identifier(s). The decentral identifier may be issued by a central or decentral identity issuer. The decentral identifier may include authentication information. Via the decentral identifier and its unique association with the data owner and chemical product data access to the chemical product data may be controlled by the data owner. This contrasts with central authority schemes, where identifiers are provided by such central authority and access to data is controlled by such central authority. Decentral in this context refers to the usage of the identifier in implementation as controlled by the data owner.

The decentral identifier may include one or more identifier(s) used in the decentral network and allowing for data exchange via the decentral network. Data exchange may include discovery of the decentral identifier for participant nodes of the decentral network, authentication of participant nodes of the decentral network and/or authorization of data transfers via a peer-to-peer communication between participant nodes of the decentral network. The decentral identifier may be associated with any participant of the product supply chain including raw chemical product supplier, intermediate chemical products manufacturer, intermediate part manufacturer, component manufacturer, component assembly manufacturer or end product manufacturer. The decentral identifier may be associated with a machine, a system, or a device used for producing the raw material, the basic substance, the chemical product, the intermediate product, the component, the component assembly or the end product, or a collection of such machine(s), device(s) and/or system(s). The decentral identifier may be associated with any participant of the recycling product chain including recycler, sorter, raw chemical product supplier, chemical product manufacturer or intermediate chemical product manufacturer.

The term “product passport” is to be understood broadly in the present case. The product passport may include data related to the product. The product passport may include a digital representation of the product data associated with the product. The product passport may comprise or be associated with a digital representation of a product. The product data may be associated with the product. The digital representation of the chemical product data may be provided to the data consuming service. The digital representation of the chemical product data may be provided by a decentral network database, a database associated with the data consuming, the data providing service associated with the data owner or combinations thereof. The digital representation may include a decentral identifier. The digital representation may include a representation for accessing product data or parts thereof. The digital representation may include a representation of product data or parts thereof. The product passport may include a chemical product passport. The product passport may include multiple passports for different physical entities in the product supply chain and/or recycling product chain. The product passport may include data related to the product data, the public key and one or more decentral identifier(s). The data related to the product data may include the digital representation of the product data. The product data may include data associated with different physical entities of the product supply chain and/or the recycling product chain.

The term “product” is to be understood broadly in the present case. The product may comprise any physical entity of the chemical product supply chain, the product supply chain and/or the recycling product chain associated with a decentral identifier to virtually identify the physical entity. Physical entities of the chemical product chain may include any chemical product to be further processed towards a product. Physical entities of the product supply chain may include a basic substance, a material, a component, a component assembly, an end product or combinations thereof. The product supply chain may include the chemical product supply chain or parts thereof or vice versa. Physical entities of the recycling product chain include an end product to be recycled, a component assembly to be recycled, a component assembly to be recycled, a material to be recycled, a basic substance to be recycled or combinations thereof. A relationship representation may be associated with a relationship between the physical entities of the product supply chain and/or the recycling product chain.

The term “recycling product or waste product” is to be understood broadly in the present case. The recycling product or waste product may comprise any physical entity of a recycling product chain associated with a decentral identifier to virtually identify the physical entity. Physical entities of the recycling product chain include an end product to be recycled, a component assembly to be recycled, a component assembly to be recycled, a material to be recycled, a basic substance to be recycled or combinations thereof. A relationship representation may be associated with a relationship between the physical entities of the product supply chain and/or the recycling product chain.

The term “chemical product passport” is to be understood broadly in the present case. The chemical product passport may include data related to the chemical product. The chemical product passport may include a digital representation of the chemical product data associated with the chemical product. The chemical product passport may comprise or be associated with a digital representation of chemical product data. The chemical product data may be associated with the chemical product. The digital representation of the chemical product data may be provided to the data consuming service. The digital representation of the chemical product data may be provided by a decentral network database, a database associated with the data consuming, the data providing service associated with the data owner or combinations thereof. The digital representation may include a representation for accessing the chemical product data or part thereof. The digital representation may include a representation of chemical product data or parts thereof. The chemical product passport may include data related to the chemical product data, the public key and the decentral identifier. The data related to the chemical product data may include the digital representation of the chemical product data.

The term “chemical product” is to be understood broadly in the present case. and the chemical product may comprise chemical products obtained from chemical reactions as well as natural chemical products. Natural chemical products encompass any chemical substance that is naturally occurring, i.e. any unprocessed chemical substance that is found in nature, such as chemicals from plants, micro-organisms, animals, the earth and the sea or any chemical substance that is found in nature and extracted using a process that does not change its chemical composition. Natural chemical products may include biologicals like enzymes as well naturally occurring inorganic or organic chemical products. Natural chemical products can be isolated and purified prior to their use or they can be used in unisolated and/or unpurified form. Chemical products obtained from chemical reactions may be any inorganic or organic chemical product obtained by reacting inorganic and/or organic chemical reactants. The inorganic and organic chemical reactants may be naturally occurring chemical products or can be chemical products obtained from chemical reactions. Chemical reactions may include any chemical reaction commonly known in the state of the art in which the reactants are converted to one or more different chemical products. Chemical reactions may involve the use of catalysts, enzymes, bacteria, etc. to achieve the chemical reaction between the reactants.

The term “chemical product data” is to be understood broadly in the present case. The chemical product data may be associated with the chemical product. The chemical product data may comprise data related to a property of the chemical product and/or data related to the use of the chemical product. Such property may be a static or a dynamic property. A static property may be a property constant over time e.g. melting point, boiling point, density, hardness, flammability our the like. A dynamic property may be a property that changes over time e.g. shelf life, pH value, color, reactivity. The property of the chemical product may include performance properties, chemical properties, such as flammability, toxicity, acidity, reactivity, heat of combustion and/or physical properties such as density, color, hardness, melting and boiling points, electrical conductivity or the like. Data related to the use of the chemical product may include data related to further processing of the chemical product, for example by using the chemical product as reactant in further chemical reaction(s) and/or data related to the use of the chemical product, for example data related to the use of the chemical product in a treatment process and/or within a manufacturing process. Chemical product data may include chemicals data, emission data, recyclate content, bio-based content and/or production data.

The term “physical entity” is to be understood broadly in the present case. The physical entity may relate to the physical embodiment of a product. The physical entity may be any product in the product supply chain, such as the chemical product supply chain, and/or the recycling product chain. The physical entity of a product may be a raw material or basic substance, a chemical product, a chemical material, a chemical formulation, a chemical mixture, a component, a component assembly, an end product or a combination thereof. The physical entity of a recycling product may be an end product to be recycled, a component assembly to be recycled, a component to be recycled, a chemical product to be recycled, a raw material to be recycled, basic substance to be recycled, a chemical material to be recycled, a chemical formulation to be recycled, a chemical mixture to be recycled or a combination thereof.

The term “product data” is to be understood broadly in the present case. The product data may be associated with the physical entity of the product. The product data may comprise data related to a property of the product and/or data related to the use of the product. Such property may be a static or a dynamic property. Product data may include emission data, recyclate content, bio-based content and/or production data. Product data may be associated with the physical entity of the chemical product. Product data may be chemical product data.

The term “recyclate content data and/or bio-based content data” is to be understood broadly in the present case. The recyclate content data and/or bio-based content data may comprise any data related to the recyclate content or the bio-based content used for providing or manufacturing a physical entity or product at any stage in the chemical supply chain such as a raw material or basic substance, chemical product or chemical material, component, component assembly or end product.

The term “emission data” is to be understood broadly in the present case. The emission data may comprise any data related to environmental footprint. The environmental footprint may refer to an entity and its associated environmental footprint. The environmental footprint may be entity specific. For instance, the environmental footprint may relate to a product, a company, a process such as a manufacturing process, a raw material or basic substance, a chemical product or material, a component, a component assembly, an end product, combinations thereof or additional entity-specific relations. Emission data may include data relating to the carbon footprint of a chemical product or a Product Carbon Footprint (PCF). Emission data may include data relating to greenhouse gas emissions e.g. released in production of the chemical product. Emission data may include data related to greenhouse gas emissions. Greenhouse gas emissions may include emissions such as carbon dioxide (CO2) emission, methane (CH4) emission, nitrous oxide (N2O) emission, hydrofluorocarbons (HFCs) emission, perfluorocarbons (PFCs) emission, sulphurhexafluoride (SF6) emission, nitrogen trifluoride (NF3) emission, combinations thereof and additional emissions.

Emission data may include data related to greenhouse gas emissions of an entities or companies own operations (production, power plants and waste incineration). Scope 2 may comprise emissions from energy production which is sourced externally. Scope 3 may comprise all other emissions along the value chain. Specifically, this may include the greenhouse gas emissions of raw materials obtained from suppliers. Product Carbon Footprint (PCF) may sum up greenhouse gas emissions and removals from the consecutive and interlinked process steps related to a particular product. Cradle-to-gate PCF may sum up greenhouse gas emissions based on selected process steps: e.g. from the extraction of resources up to the factory gate where the product leaves the company. Such PCFs may be called partial PCFs. In order to achieve such summation, each company providing any products must be able to provide the scope 1 and scope 2 contributions to the PCF for each of its products.

The term “production data” is to be understood broadly in the present case. The production data may comprise any data related to the production of a product at any stage in the chemical supply chain. Preferably production data includes chemical production data from the production of the chemical product. Production data may include monitoring and/or control data associated with the production of the product, such as a raw material or basic substance, a chemical material or chemical product, a component, a component assembly, an end product or a combination thereof. Production data may include measurement data related to a product quality at any stage in the chemical supply chain, preferably a chemical product.

The term “data owner” is to be understood broadly in the present case. The data owner may comprise any entity generating data, particularly product data. The generating node may be coupled to the entity owning physical products from or for which data, particularly, the product data, is generated. The data particularly, the chemical product data, may be generated by a third-party entity on behalf of the entity owning physical products from or for which data is generated. The data owner may be the product producer. Via the decentral identifier and its unique association with the data owner and product data access to the product data may be controlled by the data owner. The product data may be accessible for the data owner. The data owner may hence directly or indirectly own the product data. The product data may be stored in a data base of or associated with the data owner. The product data may be stored in a data base accessible by the data owner. The data owner may control access to the product data via the data providing service of the data owner. The data owner may control access to the product data.

The product data may be associated with the data owner. The data owner may be the owner of the product data or the product data owner. The product data may be stored in a data base of or under control by the data owner. In this sense, the data owner is to be construed broadly as the entity having access to the product data and controlling access by data consuming services of the decentral network to the product data.

The term “data consuming service” is to be understood broadly in the present case. The data consuming service may comprise computer-executable instructions for accessing and/or processing data, such as product data or chemical product data, associated with the data owner.

The term “data providing service” is to be understood broadly in the present case. The data providing service may comprise computer-executable instructions for providing and/or processing data, such as product data or chemical product data, associated with the data owner for accessing and/or processing by a data consuming service.

The term “digital representation(s) pointing to product data or parts thereof” is to be understood broadly in the present case. The digital representation(s) pointing to product data or parts thereof may comprise at least one interface to a data providing service. It may further include at least one interface to the data consuming service. It may include an endpoint for data exchange or sharing (resource endpoint) or an endpoint for service interaction (service endpoint), that is uniquely identified via a communication protocol. The digital representation(s) pointing to product data or parts thereof may hence be uniquely associated with the decentral identifier.

In one embodiment the request to provide the decentral identifier includes data related to the product data and/or an owner or product identifier associated with the product data owner or the product, respectively. In one embodiment the request to provide the first decentral identifier includes a product or owner identifier associated with the data owner or the product. The owner or product identifier may be a string identifier associated with an owner or product name. The owner or product identifier may be provided by a physical identifier provider, such as a bar code or a tag like a RFID tag or a QR code. Such communication can be completed via ad hoc WIFI, BLE beacon, and/or NFC. The communications between wallet apps may be performed via any available communication channel, including but not limited to, web servers, ad hoc WIFI, BLE beacon signal, NFC, a barcode or QR code scanning, etc.

In one embodiment a first decentral identifier and a second decentral identifier are provided, wherein the first decentral identifier is associated with a first physical entity and the second decentral identifier is associated with a second physical entity, and wherein the first decentral identifier is associated with the second identifier and/or vice versa according to a relationship representation. The relationship representation may include the relationship that the first physical entity is used to produce the second physical entity and/or the second physical entity is produced by using the first physical entity and/or the first physical entity is produced by using the second physical entity.

In one embodiment a first decentral identifier and a second decentral identifier are provided. The first decentral identifier may be associated with a first physical entity and the second decentral identifier may be associated with a second physical entity. The first decentral identifier may be associated with the second identifier and/or vice versa according to a relationship representation.

In one embodiment a first decentral identifier and a second decentral identifier are provided, wherein the a first decentralized identifier is associated with a first physical entity and the second decentral identifier is associated with a second physical entity, wherein the first decentral identifier associated with the first physical entity is connected to the second decentral identifier associated with the second physical entity, wherein the second physical entity is used to produce the first physical entity and/or the second physical entity is produced by using the first physical entity.

In one embodiment a first decentral identifier and a second decentral identifier are provided. The first decentral identifier may be associated with a first physical entity and the second decentral identifier may be associated with a second physical entity The first decentral identifier associated with the first physical entity may be connected to the second decentral identifier associated with the second physical entity. The second physical entity may be used to produce the first physical entity and/or the second physical entity may be produced by using the first physical entity and/or the first physical entity is produced by using the second physical entity.

In one embodiment at least one second decentral identifier associated with a second physical entity is provided, wherein the first decentral identifier is associated with the second decentral identifier and/or vice versa according to a relationship representation.

In one embodiment at least one second decentral identifier associated with a second physical entity is provided. The first decentral identifier associated with the first physical entity may be connected to the at least one second decentral identifier associated with the second physical entity. Based on the first decentral identifier, the at least one second decentral identifier and a relationship representation associated with a relationship between the at least one first physical entity and the at least one second physical entity a concatenation may be generated.

In one embodiment at least one second decentral identifier is provided. The at least one second decentral identifier may be associated with at least one second physical entity of the product supply chain, such as the chemical product supply chain, and/or the recycling product chain. A concatenation may be generated based on a relationship representation according to which the first decentral identifier is associated with the at least one second decentral identifier.

In one embodiment a product passport that further includes the at least one second decentral identifier and/or the concatenation may be generated.

In one embodiment at least one second decentral identifier is provided. The at least one second decentral identifier may be associated with the second physical entity. The first decentral identifier associated with the first physical entity may be connected to the second decentral identifier associated with the second physical entity, wherein the second physical entity is used to produce the first physical entity and/or the first physical entity is produced by using the second physical entity and/or the second physical entity is produced by using the first physical entity.

In one embodiment multiple decentral identifiers are related according to the relationship representation. The first decentral identifier may be connected to the second decentral identifier according to the relationship representation and/or vice versa. The first decentral identifier may be linked to the second decentral identifier according to the relationship representation and/or vice versa.

In one embodiment multiple second decentral identifiers are provided, wherein the multiple second decentral identifiers are related according to the relationship representation. One or more second decentral identifier(s) may be provided. The multiple or more than one second decentral identifier(s) may be connected to the first decentral identifier. The first and the second decentral identifiers may be related according to the relationship representation.

In one embodiment at least one decentral identifier is associated with multiple physical entities of the product supply chain and/or the recycling product chain. In another embodiment at least one decentral identifier is associated with each physical entity of the product supply chain and/or the recycling product chain. In another embodiment at least one decentral identifier is associated with multiple physical entities of the product supply chain and/or the recycling product chain and at least one decentral identifier is associated with one physical entity of the product supply chain and/or the recycling product chain. In one embodiment the first decentral identifier is associated with multiple physical entities of the product supply chain and/or the recycling product chain.

In one embodiment at least one decentral identifier, particularly at least one second identifier, is generated or provided per physical entity of the product supply chain and/or the recycling product chain. The decentral identifiers, particularly the first and second decentral identifiers, may be connected according to the relationship representation representing the relationship of the physical entities of the product supply chain and/or the recycling product chain.

In one embodiment at least one decentral identifier, particularly at least one first identifier, is generated for more than one physical entity of the product supply chain and/or the recycling product chain. The decentral identifiers, particularly at least one first identifier, may be connected according to the relationship representation representing the relationship of the physical entities of the product supply chain and/or the recycling product chain. More than one decentral identifier, particularly more than one second identifier, may be generated or provided each associated with one or more physical entit(y/ies) of the product supply chain and/or the recycling product chain.

In one embodiment the product passport includes or is associated with or connected to a concatenation of multiple decentral identifiers based on the relationship representation representing the relationship of the physical entities in the product supply chain and/or the recycling product chain. Multiple decentral identifiers may be more than one decentral identifier.

In one embodiment one or more hash value(s) are generated based on the relationship representation. Hash value(s) may be generated based on the concatenation of decentral identifiers. Hash value(s) may be generated for the concatenation of decentral identifiers. Hash value(s) may signify the concatenation of decentral identifiers.

The hash values may be generated based on data associated with a first decentral identifier and a second decentral identifier. The relationship representation may specify that the second physical entity may be used to produce the first physical entity and/or the first physical entity may be produced by using the second physical entity. The hash value may be generated based on a combined data set associated with the first and the second decentral identifier.

In one embodiment the product passport includes one or more second decentral identifier(s).

In one embodiment at least one decentral identifier is generated for one end product, wherein the at least one decentral identifier is associated with the physical entities covering starting entities and/or intermediate entities of the product supply chain of one end product. In other words, at least one decentral identifier may be generated for one end product and the physical entities covering starting entities and/or intermediate entities of the product supply chain may be associated with the at least one decentral identifier of the one end product. In yet other words, at least one decentral identifier may be generated for the one end product, wherein the at least one decentral identifier may be generated for association with the physical entities covering starting entities and/or intermediate entities of the product supply chain of one end product. In one embodiment at least one decentral identifier is generated for one end product and at least one decentral identifier is generated for physical entities including starting entities and/or intermediate entities of the product supply chain of one end product.

In one embodiment the at least one decentral identifier is provided from one central node or from one or more decentral nodes. The decentral identifier as generated by one central node or by one or more decentral nodes may be provided to a node generating the chemical product passport and to at least one authentication data registry, preferably accessible by the data providing service and/or the data consuming service. The authentication data registry may be a central registry such as a central file system, a centrally managed distributed database, and/or a centrally managed peer-to-peer network. The authentication data registry may be a decentral registry such as a distributed ledger, a decentralized file system, a distributed database, and/or a peer-to-peer network.

In one embodiment the product passport includes one or more authentication mechanisms associated with the decentral identifier and the data related to chemical product data. The authentication mechanism may include a token, such as private and public key infrastructure, a biometric mechanism, such as fingerprints, face recognition or voice recognition. One common public key certificate is X.509 certificate.

In one embodiment the one or more authentication mechanisms associated with the decentral identifier as generated by one central node or by one or more decentral nodes may be provided to a node generating the chemical product passport and to at least one decentral authentication data registry, preferably accessible by the data providing service and/or the data consuming service. The authentication data registry may be a central registry such as a central file system, a centrally managed distributed database, and/or a centrally managed peer-to-peer network. The authentication data registry may be a decentral registry such as a distributed ledger, a decentralized file system, a distributed database, and/or a peer-to-peer network.

In one embodiment the product passport includes one or more authorization mechanisms associated with the decentral identifier and the data related to the product data. The authorization mechanisms may include data transaction instructions or data transaction protocols, such as data usage policies, smart data contracts or more complex data processing instructions associated with data providing and/or data consuming services. Through the authentication mechanism data access by a data consuming service can be controlled in a secure manner.

In one embodiment the one or more authorization mechanisms associated with the decentral identifier as generated by one central node or by one or more decentral nodes may be provided to a node generating the product passport and to at least one decentral authentication data registry, preferably accessible by the data providing service and/or the data consuming service. In one embodiment the one or more authorization mechanisms associated with the decentral identifier as generated by one or more decentral nodes may be provided to a node generating the product passport and to at least one distributed ledger, a decentralized file system, a distributed database, and/or a peer-to-peer network, preferably accessible by the data providing service and/or the data consuming service.

In one embodiment the data related to product data includes product data or parts thereof. In one embodiment the data related to the product data includes one or more digital representation(s) pointing to product data or parts thereof. For instance, the data related to product data includes multiple digital representations pointing to distinct parts of the product data or the data related to product data includes multiple digital representations pointing to different parts of the product data, which may overlap. The representation may include an access point to the product data, a link to access product data or a service endpoint to access product data.

### BRIEF DESCRIPTION OF THE DRAWINGS

In the following, the present disclosure is further described with reference to the enclosed figures:

FIGS. 1a,b illustrate example embodiments of a centralized and a decentralized computing environment with computing nodes.

FIG. 1c illustrate an example embodiment of a distributed computing environment.

FIG. 2 shows an example of a chemical product passport including DID owner data, DID document data and decentral identity infrastructure.

FIG. 3 shows an example of a chemical product passport including ID-based data, passport data and decentral identity infrastructure.

FIG. 4 shows an example method for generation of the chemical product passport.

FIG. 5 shows an example method for using the chemical product passport to further process the chemical product associated with the chemical product passport.

FIGS. 6a, 6b show examples of authentication protocols.

FIGS. 7a, 7b illustrate the principles of cryptographic signatures.

FIG. 8 shows an example method for authorizing access to chemical product data.

FIG. 9 shows a schematic illustration of providing access to product passport of a data providing service in the International Data Space (IDS) architecture to a data consuming service.

FIG. 10 shows a schematic illustration of the authentication process between two IDS connectors.

FIG. 11 shows a schematic illustration of a usage-controlled data flow in the IDS architecture.

FIG. 12 illustrates an example of an apparatus and associated methods for generating the product passport in connection with a chemical product produced by a chemical production network.

FIGS. 13-18 show different example configurations for product passports anchored by digital identifiers.

FIGS. 19 and 20 illustrate examples of relationship representations, which may be used for generating the concatenation.

FIG. 21 illustrates an example of an apparatus for generating and using a concatenation in a decentral network.

FIG. 22 illustrates an example of a production facility producing a chemical product associated with a chemical product passport.

FIG. 23 illustrates another example of a production facility producing a chemical product associated with a chemical product passport.

FIG. 24 illustrates an example of a production system producing a chemical product associated with one or more chemical product passport(s).

FIGS. 25 to 27 illustrate examples of providing access to data via one or more chemical product passport(s).

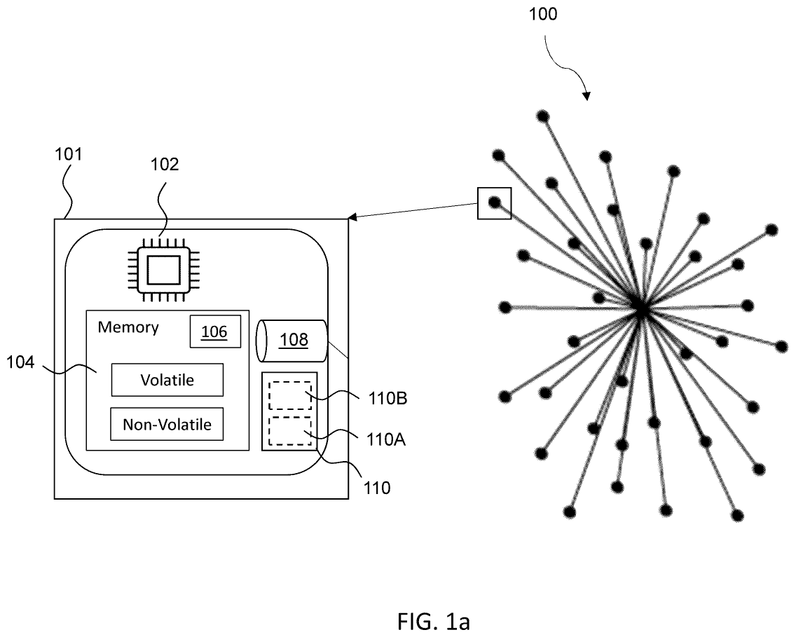

FIGS. 1a to 1c illustrate different computing environments, central, decentral and distributed. The methods, apparatuses, computer elements of this disclosure may be implemented in decentral or at least partially decentral computing environments. In particular, for data sharing or exchange in ecosystems of multiple players different challenges exist. Data sovereignty may be viewed as a core challenge. It can be defined as a natural person's or corporate entity's capability of being entirely self-determined with regard to its data. To enable this particular capability related aspects, including requirements for secure and trusted data exchange in business ecosystems, may be implemented across the chemical value chain. In particular, chemical industry requires tailored solutions to deliver chemical products in a more sustainable way by using digital ecosystems.

FIG. 1a illustrates an example embodiment of a centralized computing system 100 comprising a central computing node 101 (filled circle in the middle) and several peripheral computing nodes 101.1 to 101.n (denoted as filled circles in the periphery). The term “computing system” is defined herein broadly as including one or more computing nodes, a system of nodes or combinations thereof. The term “computing node” is defined herein broadly and may refer to any device or system that includes at least one physical and tangible processor, and a physical and tangible memory capable of having thereon computer-executable instructions that are executed by a processor. Computing nodes are now increasingly taking a wide variety of forms. Computing nodes may, for example, be handheld devices, production facilities, sensors, monitoring systems, control systems, appliances, laptop computers, desktop computers, mainframes, data centers, or even devices that have not conventionally been considered a computing node, such as wearables (e.g., glasses, watches or the like). The memory may take any form and depends on the nature and form of the computing node.

In this example, the peripheral computing nodes 101.1 to 101.n may be connected to one central computing system (or server). In another example, the peripheral computing nodes 101.1 to 101.n may be attached to the central computing node via e.g. a terminal server (not shown). The majority of functions may be carried out by, or obtained from the central computing node (also called remote centralized location). One peripheral computing node 101.n has been expanded to provide an overview of the components present in the peripheral computing node. The central computing node 101 may comprise the same components as described in relation to the peripheral computing node 101.n.

Each computing node 101, 101.1 to 101.n may include at least one hardware processor 102 and memory 104. The term “processor” may refer to an arbitrary logic circuitry configured to perform basic operations of a computer or system, and/or, generally, to a device which is configured for performing calculations or logic operations. In particular, the processor, or computer processor may be configured for processing basic instructions that drive the computer or system. It may be a semi-conductor based processor, a quantum processor, or any other type of processor configures for processing instructions. As an example, the processor may comprise at least one arithmetic logic unit (“ALU”), at least one floating-point unit (“FPU)”, such as a math coprocessor or a numeric coprocessor, a plurality of registers, specifically registers configured for supplying operands to the ALU and storing results of operations, and a memory, such as an L1 and L2 cache memory. In particular, the processor may be a multicore processor. Specifically, the processor may be or may comprise a Central Processing Unit (“CPU”). The processor may be a (“GPU”) graphics processing unit, (“TPU”) tensor processing unit, (“CISC”) Complex Instruction Set Computing microprocessor, Reduced Instruction Set Computing (“RISC”) micro-processor, Very Long Instruction Word (“VLIW”) microprocessor, or a processor implementing other instruction sets or processors implementing a combination of instruction sets. The processing means may also be one or more special-purpose processing devices such as an Application-Specific Integrated Circuit (“ASIC”), a Field Programmable Gate Array (“FPGA”), a Complex Programmable Logic Device (“CPLD”), a Digital Signal Processor (“DSP”), a network processor, or the like. The methods, systems and devices described herein may be implemented as software in a DSP, in a micro-controller, or in any other side-processor or as hardware circuit within an ASIC, CPLD, or FPGA. It is to be understood that the term processor may also refer to one or more processing devices, such as a distributed system of processing devices located across multiple computer systems (e.g., cloud computing), and is not limited to a single device unless otherwise specified.

The memory 104 may refer to a physical system memory, which may be volatile, non-volatile, or a combination thereof. The memory may include non-volatile mass storage such as physical storage media. The memory may be a computer-readable storage media such as RAM, ROM, EEPROM, CD-ROM, or other optical disk storage, magnetic disk storage, or other magnetic storage devices, or any other physical and tangible storage medium which can be used to store desired program code means in the form of computer-executable instructions or data structures and which can be accessed by the computing system. Moreover, the memory may be a computer-readable media that carries computer-executable instructions (also called transmission media). Further, upon reaching various computing system components, program code means in the form of computer-executable instructions or data structures can be transferred automatically from transmission media to storage media (or vice versa). For example, computer-executable instructions or data structures received over a network or data link can be buffered in RAM within a network interface module (e.g., a “NIC”), and then eventually transferred to computing system RAM and/or to less volatile storage media at a computing system. Thus, it should be understood that storage media can be included in computing components that also (or even primarily) utilize transmission media.

The computing nodes 101, 101.1 . . . 101.n may include multiple structures 106 often referred to as an “executable component or computer-executable instructions”. For instance, memory 104 of the computing nodes 101, 101.1 . . . 101.n may be illustrated as including executable component 106. The term “executable component” may be the name for a structure that is well understood to one of ordinary skill in the art in the field of computing as being a structure that can be software, hardware, or a combination thereof or which can be implemented in software, hardware, or a combination. For instance, when implemented in software, one of ordinary skill in the art would understand that the structure of an executable component include software objects, routines, methods, and so forth, that is executed on the computing nodes 101, 101.1 . . . 101.n, whether such an executable component exists in the heap of a computing node 101, 101.1 . . . 101.n, or whether the executable component exists on computer-readable storage media. In such a case, one of ordinary skill in the art will recognize that the structure of the executable component exists on a computer-readable medium such that, when interpreted by one or more processors of a computing node 101, 101.1 . . . 101.n (e.g., by a processor thread), the computing node 101, 101.1 . . . 101n is caused to perform a function. Such a structure may be computer-readable directly by the processors (as is the case if the executable component were binary). Alternatively, the structure may be structured to be interpretable and/or compiled (whether in a single stage or in multiple stages) so as to generate such binary that is directly interpretable by the processors. Such an understanding of example structures of an executable component is well within the understanding of one of ordinary skill in the art of computing when using the term “executable component”. Examples of executable components implemented in hardware include hardcoded or hard-wired logic gates, that are implemented exclusively or near-exclusively in hardware, such as within a field-programmable gate array (FPGA), an application-specific integrated circuit (ASIC), or any other specialized circuit. In this description, the terms “component”, “agent”, “manager”, “service”, “engine”, “module”, “virtual machine” or the like are used synonymous with the term “executable component.

The processor 102 of each computing node 101, 101.1 . . . 101.n direct the operation of each computing node 101, 101.1 . . . 101.n in response to having executed computer-executable instructions that constitute an executable component. For example, such computer-executable instructions may be embodied on one or more computer-readable media that form a computer program product. The computer-executable instructions may be stored in the memory 104 of each computing node 101, 101.1 . . . 101.n. Computer-executable instructions comprise, for example, instructions and data which, when executed at a processor 101, cause a general purpose computing node 101, 101.1 . . . 101.n, special purpose computing node 101, 101.1 . . . 101.n, or special purpose processing device to perform a certain function or group of functions. Alternatively or in addition, the computer-executable instructions may configure the computing node 101, 101.1 . . . 101.n to perform a certain function or group of functions. The computer executable instructions may be, for example, binaries or even instructions that undergo some translation (such as compilation) before direct execution by the processors, such as intermediate format instructions such as assembly language, or even source code.

Each computing node 101, 101.1 . . . 101.n may contain communication channels 108 that allow each computing node 101.1 . . . 101.n to communicate with the central computing node 101, for example, a network (depicted as solid line between peripheral computing nodes and the central computing node in FIG. 1a). A “network” may be defined as one or more data links that enable the transport of electronic data between computing nodes 101, 101.1 . . . 101.n and/or modules and/or other electronic devices. When information is transferred or provided over a network or another communications connection (either hardwired, wireless, or a combination of hardwired or wireless) to a computing node 101, 101.1 . . . 101.n, the computing node 101, 101.1 . . . 101.n properly views the connection as a transmission medium. Transmission media can include a network and/or data links which can be used to carry desired program code means in the form of computer-executable instructions or data structures and which can be accessed by a general-purpose or special-purpose computing nodes 101, 101.1 . . . 101.n. Combinations of the above may also be included within the scope of computer-readable media.

The computing node(s) 101, 101.1 to 101.n may further comprise a user interface system 110 for use in interfacing with a user. The user interface system 110 may include output mechanisms 110A as well as input mechanisms 110B. The principles described herein are not limited to the precise output mechanisms 110A or input mechanisms 110B as such will depend on the nature of the device. However, output mechanisms 110A might include, for instance, displays, speakers, displays, tactile output, holograms and so forth. Examples of input mechanisms 110B might include, for instance, microphones, touchscreens, holograms, cameras, keyboards, mouse or other pointer input, sensors of any type, and so forth.

FIG. 1b illustrates an example embodiment of a decentralized computing environment 100′ with several computing nodes 101.1′ to 101.n′ denoted as filled circles. In contrast to the centralized computing environment 100 illustrated in FIG. 1a, the computing nodes 101.1′ to 101.n′ of the decentralized computing environment are not connected to a central computing node 101 and are thus not under control of a central computing node. Instead, resources, both hardware and software, may be allocated to each individual computing node 101.1′ . . . 101.n′ (local or remote computing system) and data may be distributed among various computing nodes 101.1′ . . . 101.n′ to perform the tasks. Thus, in a decentral system environment, program modules may be located in both local and remote memory storage devices. One computing node 101′ has been expanded to provide an overview of the components present in the computing node 101′. In this example, the computing node 101′ comprises the same components as described in relation to FIG. 1a.

FIG. 1c illustrates an example embodiment of a distributed computing environment 103. In this description, “distributed computing” may refer to any computing that utilizes multiple computing resources. Such use may be realized through virtualization of physical computing resources. One example of distributed computing is cloud computing. “Cloud computing” may refer a model for enabling on-demand network access to a shared pool of configurable computing resources (e.g., networks, servers, storage, applications, and services). When distributed, cloud computing environments may be distributed internationally within an organization and/or across multiple organizations. In this example, the distributed cloud computing environment 103 may contain the following computing resources: mobile device(s) 114, applications 116, databases 118, data storage 120 and server(s) 122. The cloud computing environment 103 may be deployed as public cloud 124, private cloud 126 or hybrid cloud 128. A private cloud 124 may be owned by an organization and only the members of the organization with proper access can use the private cloud 126, rendering the data in the private cloud at least confidential. In contrast, data stored in a public cloud 126 may be open to anyone over the internet. The hybrid cloud 128 may be a combination of both private and public clouds 124, 126 and may allow to keep some of the data confidential while other data may be publicly available.

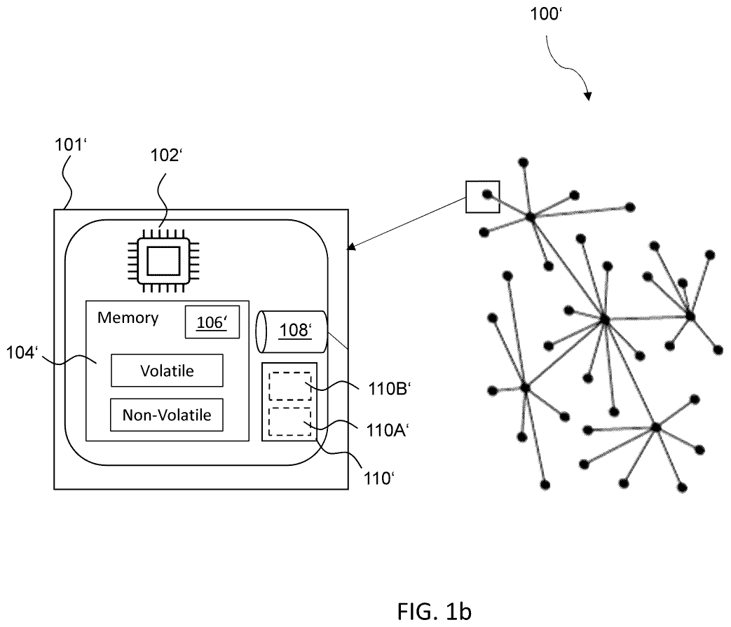

FIG. 2 shows an example of ID-based owner data, ID-based passport data and a decentralized identity manager.

The ID may be a decentralized ID (DID). The ID-based passport data may be a DID document associated with the DID. The ID-based owner data may include an ID associated with a subject such as product data or chemical product data and may include authentication mechanisms. The ID-based owner data may include owner data that is electronically owned and controlled by the DID owner. In this context electronically owned may refer to data that is stored in an owner repository or wallet. Such data may be securely stored and/or managed on an organizational server or client device. The ID-based owner data may include a DID, a private key and a public key. The ID-based owner may own and control the DID that represents an identity associated with the DID subject, a private key and public key pair that are associated with the DID. DID may be understood as an identifier and authentication information associated with or uniquely linked to the identifier.

The DID subject may be a raw material, a basic substance, a chemical product, an intermediate product, a component, a component assembly or an end product. The DID subject may be a machine, a system, or a device used for producing the raw material, the basic substance, the chemical product, the intermediate product, the component, the component assembly or the end product, or a collection of such machine(s), device(s) and/or system(s). The DID owner may be a supply chain participant or a manufacturer such as a chemical manufacturer producing chemicals. The DID owner may be an upstream participant in the supply chain of the chemical manufacturer such as a supplier that supplies raw chemical products or precursors to produce chemicals. The DID owner may be a downstream participant in the supply chain of the chemical manufacturer such as a customer that consumes chemicals to produce the intermediate product, the component, the component assembly or the end product. The DID owner may be any participant of the supply chain including raw chemical product supplier, intermediate chemical products manufacturer, intermediate part manufacturer, component manufacturer, component assembly manufacturer or end product manufacturer.

The DID may be any identifier that is associated with the DID subject and/or the DID owner. Preferably, the identifier is unique to the DID subject and/or DID owner. The identifier may be unique at least within the scope in which the DID is anticipated to be in use. The identifier may be a locally or globally unique identifier for the raw material, the basic substance, the chemical product, the intermediate product, the component, the component assembly, the end product or a collection thereof; the machine, the system, or the device used for producing the raw material, the basic substance, the chemical product, the intermediate product, the component, the component assembly or the end product, or the collection of such machine(s), device(s) and/or system(s); the chemical manufacturer producing chemicals, the upstream participant in the supply chain of the chemical manufacturer, the downstream participant in the supply chain of the chemical manufacturer or a collection thereof; any participant of the supply chain including raw chemical product supplier, intermediate chemical products manufacturer, intermediate part manufacturer, component manufacturer, component assembly manufacturer or end product manufacturer or a collection thereof.

The DID may be a Uniform Resource Identifier (URI) such as a Uniform Resource Locator (URL). The DID may be an Internationalized Resource Identifier (IRI). The DID may be a random string of numbers and letters for increased security. In one embodiment, the DID may be a string of 128 letters and numbers e.g. according to the scheme did: method name: method specific-did such as did:example:ebfeb1f712ebc6f1c276e12ec21. The DID may be decentralized independent of a centralized, third party management system and under the control of the DID owner.

The DID document 16 may be associated with the DID. Accordingly, the DID document 16 may include a reference to the DID, which is associated with the DID subject that is described by the DID document. The DID document 16 may also an authentication information such as the public key. The public key may be used by third-party entities that are given permission by the DID owner/subject 14 to access information and data owned by the DID owner/subject 14. The public key may also be used for verifying that the DID owner 14, in fact, owns or controls the DID. The DID document 16 may include authentication information, authorization information e.g. to authorize third party entities to read the DID document or some part of the DID document 16 e.g. without giving the third party the right to prove ownership of the DID.

The DID document 16 may include one or more representations that digitally link to the product data or chemical product data, e.g. by way of service endpoints. A service endpoint may include a network address at which a service operates on behalf of the DID owner. In particular, the service endpoints may refer to services of the DID owner that give access to product data or chemical product data. Such services may include services to read or analyze product data or chemical product data. Chemical product data may include chemical product declaration data, chemical product safety data, certificate of analysis data, emission data, product carbon footprint data, product environmental footprint data, chemical product specification data, product information, technical application data, production data or combinations thereof.

The DID document 16 may include various other information such metadata specifying when the DID document 16 was created, when it was last modified and/or when it expires.

The DID and DID document may be associated with a data registry node such as a centralized data service system or a decentralized data service system, e.g. a distributed ledger or blockchain 10. Possible blockchain systems include Quorum, Hyperledger Fabric. The distributed ledger or blockchain 10 may be used to store a representation of the DID 14 that points to the DID document 16. A representation of the DID may be stored on distributed computing nodes of the distributed ledger or blockchain 10. For example, DID hash may be stored on multiple computing nodes of the distributed ledger 10 and point to the location of the DID document 16. In some embodiments, the DID document 16 may be stored on the distributed ledger. Alternatively, in other embodiments the DID document 16 may be stored in a data storage (not illustrated) that is associated with the distributed ledger or blockchain 10.

The distributed ledger or blockchain 10 may be any decentralized, distributed network that includes various computing nodes that are in communication with each other. For example, the distributed ledger 10 may include a first distributed computing node 12.1, a second distributed computing node 12.2, a third distributed computing node 12.3, and any number of additional distributed computing node as illustrated by 12.4, 12.5. The distributed ledger or blockchain 10 may operate according to any known standards or methods for distributed ledgers. Examples of conventional distributed ledgers that correspond to the distributed ledger or blockchain 10 include, but are not limited to, Bitcoin [BTC], Ethereum, and Litecoin.

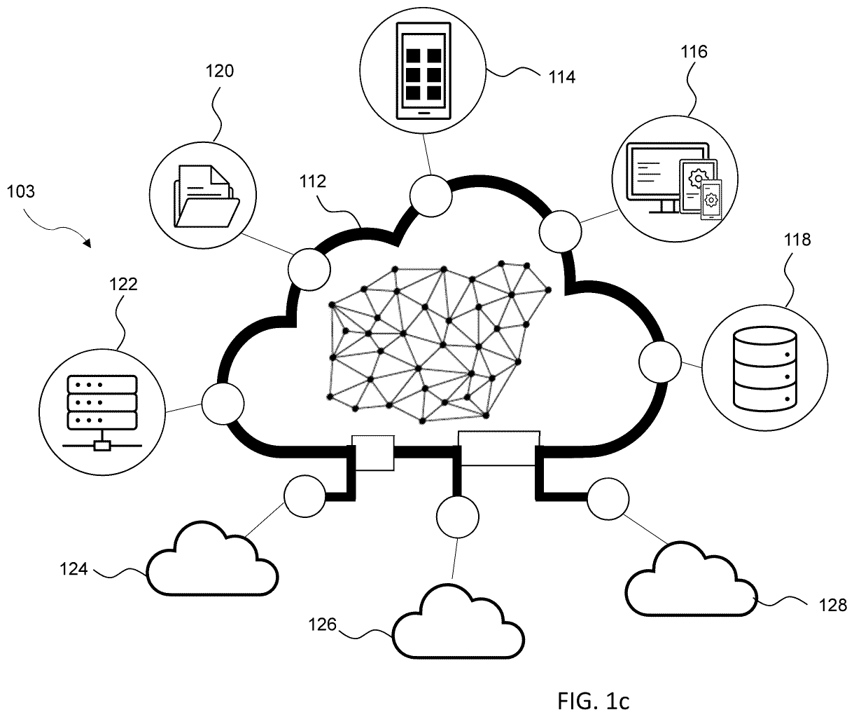

FIG. 3 show an example of ID-based certificate data, ID-based passport data and an identity manager.

In contrast to the example of FIG. 2, the example of FIG. 3 is certificate based. ID-based certificate data may include authentication data of the certificate owner and the certificate issuer. For example, a cryptographic signature from the issuer may bind the public key of the data owner to the ID. The ID may be a unique ID (such as UID) as described in relation to the DID of FIG. 2. The certificate may be a X.509 certificate such as X509v3. The ID-based passport data may be associated with the data source of the data owner. The ID-based passport data may include an ID, authentication data and endpoints associated with product data or chemical product data. Such endpoints may include any digital representation connecting to the data source. The data source may provide product data and/or chemical product data.

In this certificate-based example, the ID-based passport data includes one or more certificate(s) associated with the data owner. The certificates may be associated with an identity manager including e.g. a certificate issuing service and/or a dynamic provisioning service providing dynamic attribute tokens (e.g. OAuth Access Tokens). The information required to verify the certificates are provided via an authentication registry associated with the certificate issuing service and/or a dynamic provisioning service. For instance, in the IDSA Reference Architecture Model, Version 3.0 of April 2019, a connector associated with the data owner, a Certification Authority (CA), a Dynamic Attribute Provisioning Service (DAPS) and a connector associated with the data consumer service are used to verify the identity prior to performing a data exchange (not shown). For this purpose, such connectors include one or more certificate(s) such as X.509 certificate(s). This way the connector possesses a unique identifier embedded in a X.509 certificate that identifies the connector instance.

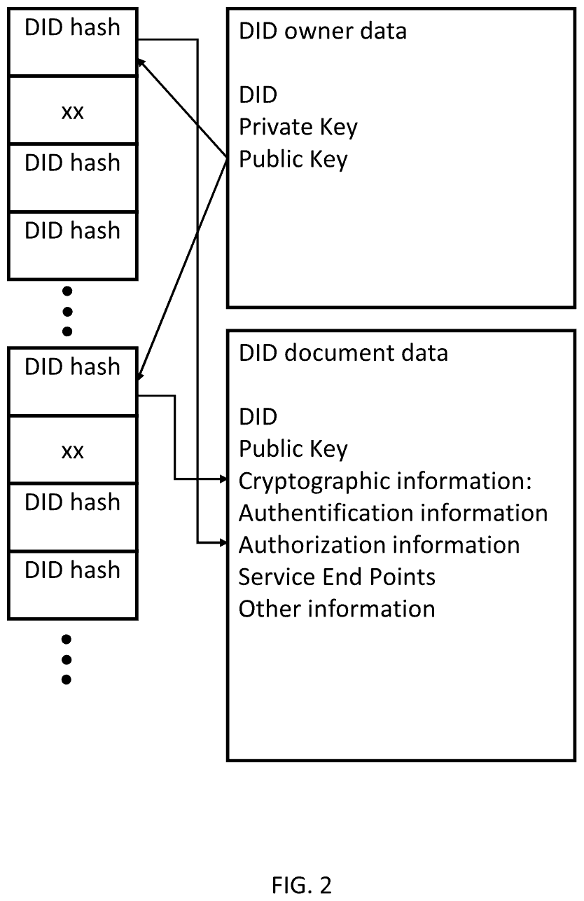

FIG. 4 shows an example method for generation of the chemical product passport.

For chemical passport generation, a request to provide a decentral identifier associated with a data owner and chemical product data may be provided. A computing node (that acts as a DID owner's management module, user agent, ID hub and/or certification issuer) may receive an indication to generate a decentral identifier. The indication may include providing at least one authentication mechanism or selecting at least one of multiple authentication mechanisms. In response to the request, the chemical product passport including the decentral identifier and data related to the chemical product data may be generated. An authentication mechanism may be selected or provided. The decentral identifier and data related to the authentication mechanism may be generated or provided. The digital representation connected to the chemical product data may be provided. Based on the decentral identifier, data related to the authentication mechanism and the digital representation connected to the chemical product data, the chemical product passport may be generated.

The chemical product passport may be provided for access by a data consuming service controlled by a data providing service associated with a data owner. The chemical product passport may include at least (1) data related to the decentral identifier and (2) data related to the select at least one authentication mechanism. Next, at least a portion of data contained in the chemical product passport may be propagated to a authentication data registry such as a distributed ledger. The chemical product passport may further include data related to the chemical product data.

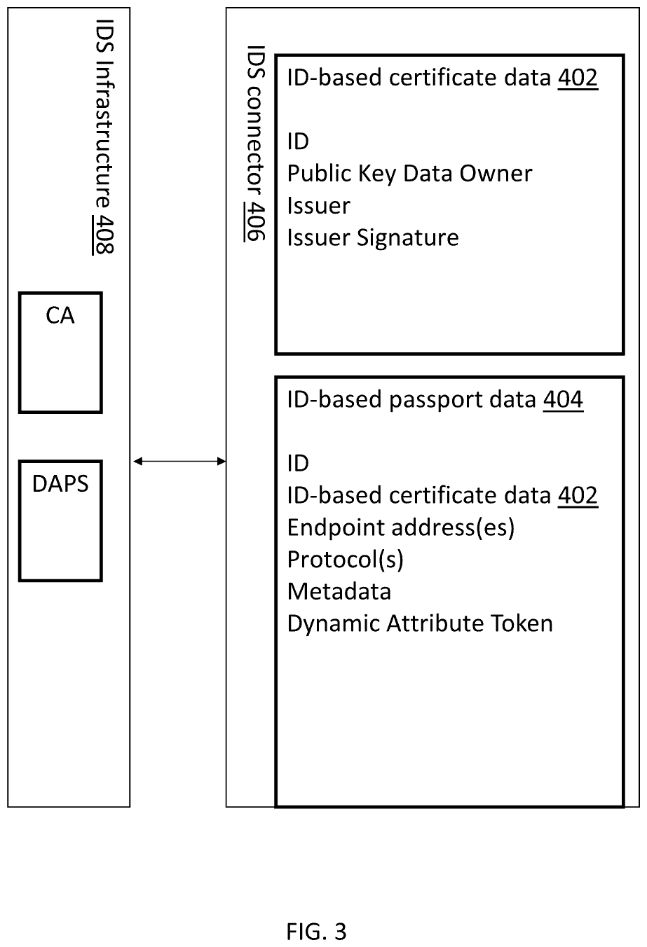

FIG. 5 shows an example method for using the chemical product passport to further process the chemical product associated with the chemical product passport.

For using the chemical product passport, an indication to access the chemical product data associated with a decentral identifier of the chemical product passport may be received. The chemical product passport may be structured as lined out in FIGS. 2 and 3. The chemical product passport may be generated as lined out in FIG. 5.

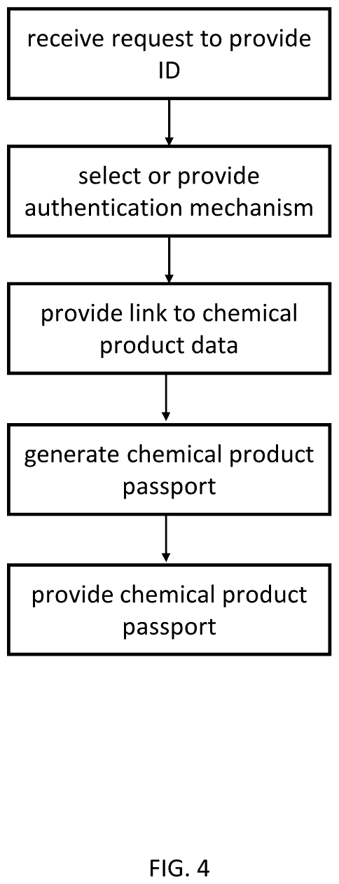

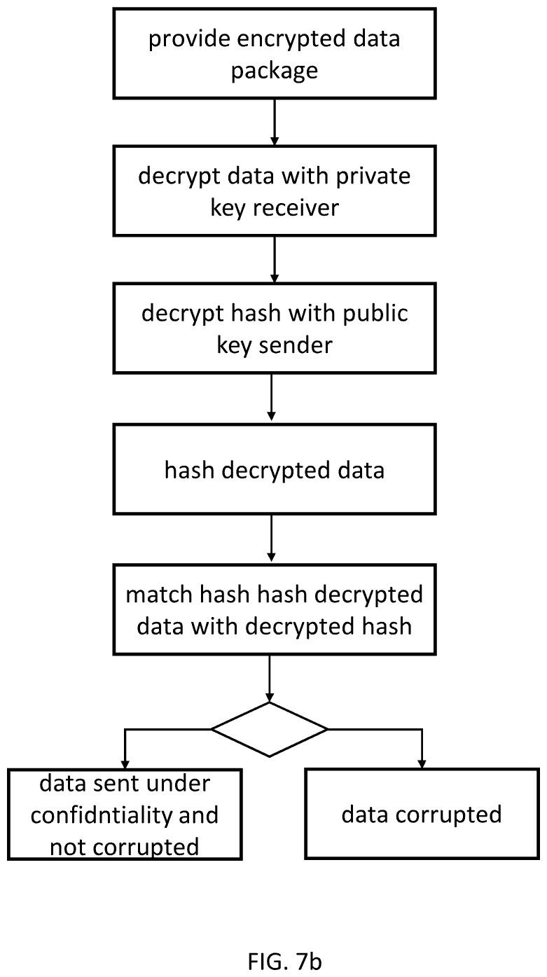

Before access may be provided to the chemical product data the request may be authenticated. In particular, the data consuming service requesting to access the chemical process data and/or the data provider service providing access to the chemical process data may be authenticating. Such authentication may be based on the decentral identity and the data related to the authentication mechanism. The authentication may be performed through different communication patterns, which will be lined out in more detail in FIGS. 6 and 10.

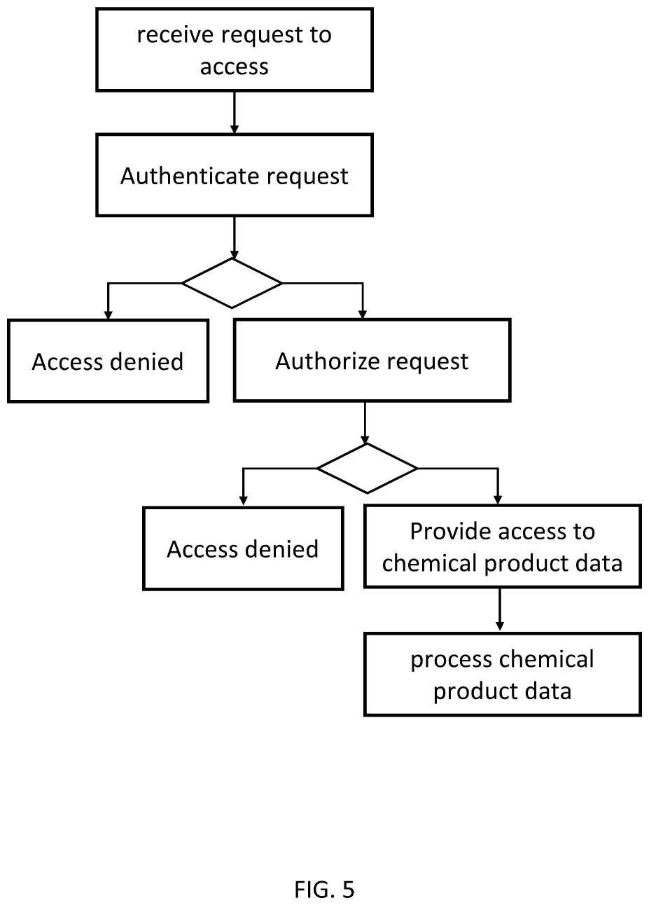

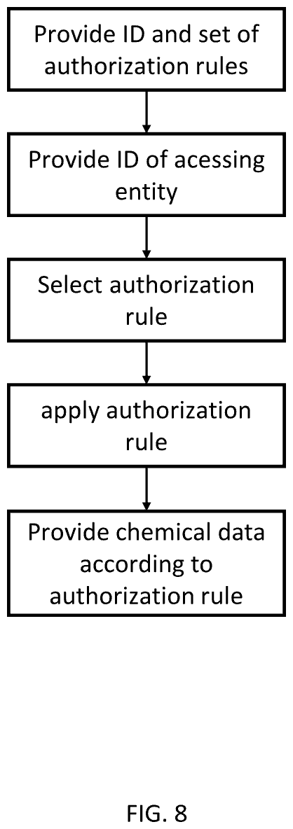

If the authentication fails, access to the chemical product data may be denied. If the authentication is valid, an authorization step may follow. Such authorization may be based on the decentral identity and the data related to the authorization rules. The authorization may be performed through different communication patterns, which will be lined out in more detail in FIGS. 7 and 11.

If the authorization fails, access to the chemical product data may be denied or access may be adapted. In particular, the authorization as requested may be adapted to be in line with the applicable authorization rules. If the authorization is valid, access to the chemical product data may be granted according to the authorization rules as requested. Such access to chemical product data associated with the decentral identifier may be provided using the representation embodied in the chemical product passport.

FIG. 6 shows an example method for authentication to access of chemical product data.

In the process of authentication, various communication patterns may be implemented to verify identities. FIG. 6a illustrates one example communication pattern that may occur between a data providing service and a data consuming service. In this case, the data providing service may act as verifying entity and no separate service may be used for authentication.

The data consuming service may request a service from the data providing service. The request may include the data consuming service decentral identifier such as a DID.

In response to the request, the data providing service may access a registry such as a central or decentral authentication registry to retrieve data related to the authentication mechanism(s) associated with the ID. For instance, the central authentication registry may provide data related to authentication mechanism via an authentication service issuing access token. Further for instance, the decentral authentication registry may provide data related to authentication mechanism by generating a request token. Data related to authentication mechanism may include a public key of the data consuming service.

Based on the retrieved data related to the authentication mechanism(s), the data providing service may generate an authentication request (corresponding for example to authentication request tokens or dynamic attribute tokens). The authentication request may be generated based on a public key of the data consuming service and/or the private key of the data provider service. The generated authentication request may be sent to the data consuming service.

Based on the received authentication request, the data consuming service may generate authentication data for responding to the authentication request. The generated authentication data may be sent back to the data providing service.

Receiving the response including the authentication data from data consuming service, the data providing service may then validate the authentication data. In response to the validation, the data providing service may grant or deny the service request of the data consuming service.

FIG. 6b illustrates yet another communication pattern that occurs amongst data provider service, an authentication service, and data consumer service.

First, the data consumer service may request a service or initiates a communication with data provider service. The request may include the decentral identifier such as a DID of the data consumer service.

Receiving the request, the data provider service may access a distributed ledger retrieve one or more authentication mechanism(s) associated with the ID. Based on the retrieved authentication mechanisms(s), the service provider may generate an authentication request.

Here, the at least one of the retrieved authentication mechanism(s) may be provided via the authentication service. As such, in some embodiments, the generated authentication request may be sent to the authentication service directly. Receiving the authentication request from the data provider service, the authentication service may generate the authentication data. The authentication data generated by the authentication service may be sent to the data consumer service.

Data consuming service then, in turn, may pass on the authentication data to the data provider service. Receiving the authentication data, the data provider service may then validate the authentication data. In response to the validation, the data providing service may grant or deny the service request of the data consuming service.

Alternatively, in some embodiments, after the data provider service may generate an authentication request, the data provider service may send the authentication requests to data consumer service. The data consumer service may pass on the authentication request to the authentication service.

Further, after the authentication service may generate the authentication data, in some embodiments, the authentication service merely contacts the data consumer service to notify the receipt of the authentication request and to obtain consent. When the data consumer service receives the notification, the data consumer service consents and sends the consent back to the authentication service. Receiving the consent, the authentication service then sends the authentication data directly to the data provider service.

Finally, in many transactions, the authentication may be mutually performed by both parties. In such a mutual authentication situation, each involved party is both a subject entity and a verifying entity. Data consumer service and data provider service have control over their ID. At the beginning, services exchange their ID. Next, each of the services accesses a distributed ledger to obtain each other's authentication mechanism(s). Each service then generates its own authentication request based on the other ID's authentication method(s). The generated authentication data is then sent to the other service. Receiving each other's authentication data, each service validates the received authentication data. Based on the validation results, the services may then perform additional communications, e.g. one service may grant or deny the service request of the other service.

FIGS. 6a and 6b only show examples of authentication protocols. Also, although the communication arrows were discussed in a certain order or illustrated in a sequence of communications, no particular ordering is required unless specifically state, or required because a communication is dependent on another communication being completed prior to the communication being transmitted.

FIGS. 7a and 7b illustrate the principle of cryptographic signature as it may be used for example in authentication mechanisms based on a private and public key pair. Such processes include cryptographic mechanisms using the key pair and/or hashing functions.

FIG. 7a illustrates one example of an encryption process. In a first step the subject data such as a JWT request token may be provided. Such data may include the public key of the receiver. Further the private key of the sender may be provided.

In a second step the data or parts of the data may be transformed through a hash function. Hash functions may map multiple inputs to exactly one output. Known hash functions are for instance SHA256, MD5, Bcyrpt or RIPEMD.

In a third step the hashed data or the hash may be encrypted with the private key of the sender. Such encryption of the data or hashed data ensures that the data is provided by the sender as the legitimate owner and not a non-legitimate third-party. This process of encryption with the private key of the sender may also be referred to as signature. The additional use of a hashing function allows to reduce the data package size to be transmitted over the network.

In a fourth step the data and encrypted hash are encrypted with the public key of the receiver. In a fifth step the encrypted data package including the data and the signature are provided for transmission to the sender.

FIG. 7b illustrates one example of a decryption process for an encrypted data package of FIG. 7a. In a first step the encrypted data package may be provided. Further the public key of the sender and the private key of the receiver may be provided.

In a second step the data package may be decrypted with the private key of the receiver. This way confidentiality of the data transmission can be ensured.

In a third step the hash of the decrypted data package may be decrypted with the public key of the sender. This way the signature of the sender can be resolved to ensure legitimate sender. In a fourth step the decrypted data from the decrypted data may be hashed. In a fifth step the hashes from the decrypted hash and the decrypted data may be matched. If the hashes match the data was sent under confidentiality and is not corrupted. If the hashes do not match the data is corrupted.

FIGS. 7a and 7b only illustrate the principle of authentication mechanisms based on cryptographic signatures. Multiple different authentication mechanisms exist and multiple variations can be implemented. Further examples of authentication mechanisms include: Biometric authentication like Touch ID or Face ID, FIDO Security keys, push authentication, biometrics and device binding mechanisms, magic links, any combinations thereof or any other password-less authentication mechanism known to the person skilled in the art.

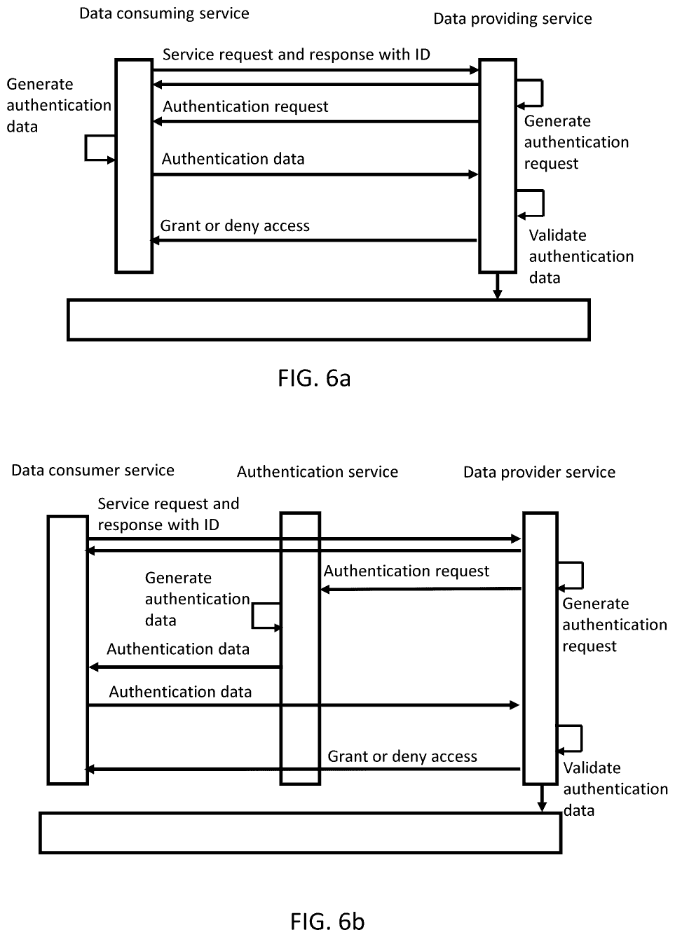

FIG. 8 shows an example method for authorizing access to chemical product data.

In a first step a first decentral identifier and a set of authorization rules for the chemical product data associated with the first decentral identifier are provided. The set of authorization rules may include usage instructions defining usage policies for entities accessing the chemical product data associated with the decentral identifier. The set of rules may include one or more local rules that are specific to a particular location. The one or more local rules may be based on a location of where the decentral identifier was generated, where the data providing service was implemented, where the data consuming service was implemented or a combination thereof.

The one or more sets of local rules may be based on a location provided by the data providing service or the data providing service. The location may refer to a jurisdiction and the local rules may be associated with legal requirements related to the supply of chemicals or chemical materials. For instance access to chemicals data may be provided via an authorization rule that may include jurisdictional or local rules. Chemicals data may include certificate of analysis data associated with laboratory measurement data acquired from a sample of the raw material, the chemical product, the component, the component assembly, or the end product and/or combinations thereof. The certificate of analysis data may by associated with more than one raw material or chemical product such as those used to manufacture the component. The certificate of analysis data may by associated with more than one raw material or chemical product such as those used to manufacture multiple components to assemble component assembly or end product. chemicals data include chemical product safety data associated with hazards of substances or mixtures of the physical entity of the product such as the raw material, the chemical product, the component, the component assembly, or the end product and/or combinations thereof. The chemical product safety data may by associated with more than one raw material or chemical product such as those used to manufacture the component. The chemical product safety data may by associated with more than one raw material or chemical product such as those used to manufacture multiple components to assemble component assembly or end product. Chemicals data may include chemical product declaration data associated with physical entity of a product, such as the raw material, the chemical product, the component, the component assembly, or the end product and/or combinations thereof. The chemical product declaration data may by associated with more than one raw material or chemical product such as those used to manufacture the component. The chemical product declaration data may by associated with more than one raw material or chemical product such as those used to manufacture multiple components to assemble component assembly or end product.

In a second step a second decentral identifier or data related to the second decentral identifier of the accessing entity is provided. Based on the second decentral identifier or data related to the second decentral identifier of the accessing entity, the authorization rule for the chemical product data associated with the first decentral identifier is selected. The authorization rule may include computer-executable instructions to allow, deny or modify chemical product data. The authorization rule may relate to each data point of the chemical product data or sets or classes of chemical product data. The selected authorization rule may be stored for application to the chemical product data. Such authorization rule may be applied before or on data transaction. The selected authorization rule may be bound to the chemical product data, individual data points or classes of chemical product data for application to the chemical product data. The product data may include data related to different classes of product data, such as chemical product data. For instance, the data chemical product data may itself include different classes of chemical product data. In one embodiment at least one class of chemical product data includes chemicals data e.g. data required by regulation or regulatory data for chemicals.

The selected authorization rule may be applied to the chemical product data associated with the first decentral identifier. The selected authorization rule may be applied prior to access of the chemical product data. The selected authorization rule may be applied during run-time on access of the chemical product data.

The chemical product data associated with the first decentral identifier is provided according to the selected authorization rule. The authorization rule may for instance may include local rules that are specific to a particular location, wherein the location is associated with a jurisdiction and the local rule for the location is associated with legal requirements related to the supply of chemicals. The set of authorization rules may include at least one regulatory instruction configured to provide access to chemicals data relating to regulatory requirements for the supply of chemicals. The provided set of authorization rules may be related to decentral identifiers of accessing entities. the authorization rule may include computer-executable instructions to allow access to chemical product data associated with the first decentral identifier, deny access to chemical product data associated with the first decentral identifier, to modify access to chemical product data associated with the first decentral identifier or to modify chemical product data associated with the first decentral identifier. The authorization rule may relate to each data point of the chemical product data or classes of chemical product data, wherein the selected authorization rule is bound to the chemical product data, classes of chemical product data, individual data points or combinations thereof. The set of authorization rules may include one or more of prescribed rules relating to obligations of the data consuming service associated with the second decentral identifier. The set of authorization rules includes one or more of prescribed rules relating to emission data, production data, recyclate content data, bio-based content data, provenance data, labour conditions data or combinations thereof. The set of authorization rules may include one or more processing rules relating to the processing of emission data, production data, recyclate content data, bio-based content data, provenance data, labour conditions data or combinations thereof by a data consuming service associated with decentral identifiers of accessing entities. The set of authorization rules includes one or more aggregation rules relating to bill of material data provided by a data providing service of a supplier or raw material data provided by a data providing service of a supplier.

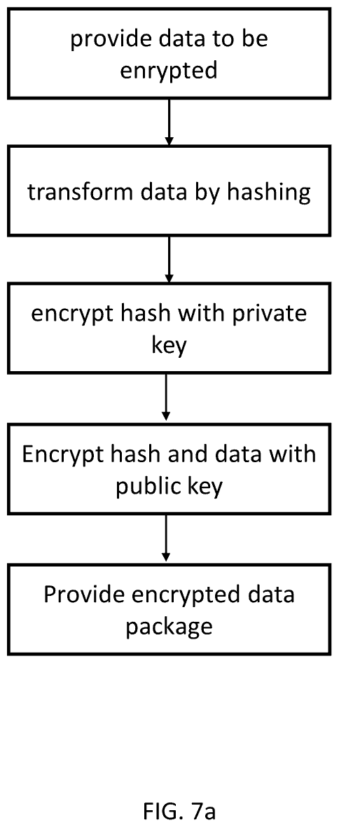

FIG. 9 shows a schematic illustration of providing a chemical product passport from a data providing service via the IDS architecture.

The data providing service may provide chemical product data to a data consuming service. The following IDS components may be executable components in the “IDS infrastructure” shown in FIG. 8: Data Connector, App Store, Identity Provider, Vocabulary Provider, Broker, Clearing House. Data may be exchanged between an interface of a data owner and an interface of a data user via a data providing service and a data consuming service. The data providing service and the data consuming service are each connected to the data connector to allow a secure and trusted data exchange. The data provided by the interface of the data owner may be associated with authorization mechanisms such as usage policies specifying authorization rules such as data usage rules. Exchange or sharing of data may be conducted according to predefined authorization mechanisms as described in relation to FIG. 7. The data provided by the interface of the data owner via the data providing service may be accessed by the interface of the data user according to the usage policies attached to the data provided by the interface of the data owner.

A participant acting as Data Owner may assume the role of the Data Provider. However, there may be cases in which the Data Provider may not coincide with the Data Owner (e.g., if the data is technically managed by a different entity than the Data Owner, such as in the case of a company using an external service to provide data, or if data management activities are handed over to a data trustee). In cases in which the Data Owner does not act as the Data Provider at the same time, the only activity of the Data Owner is to authorize a Data Provider to make its data available to be used by the interface of the Data Consumer.

The Data Provider interface makes data available for being shared or exchanged between a Data Owner and a Data Consumer. To facilitate a data request from a Data Consumer interface, the Data Provider interface may provide metadata, such as a description of datasets e.g. the syntax, serialization and/or semantics of data sources, a description of the provider or a description of consumers, to a Broker Service Provider (see below). A Broker Service Provider is not necessarily required for a Data Consumer interface and a Data Provider interface to establish a connection.

Exchanging or sharing data with a Data Consumer interface needs not necessarily be the only activity of the Data Provider. The Data Provider interface may log the details of the successful (or unsuccessful) completion of transactions at a Clearing House (see below). The data provider interface may facilitate billing or resolve a conflicts. Furthermore, the Data Provider interface may use Data Apps to check, enrich or transform the data.

The Data Consumer interface may receive data from a Data Provider interface. The Data Consumer interface may the mirror entity of the Data Provider interface. The executable components of the Data provider interface may be mirrored by the Data Consumer interface. Before the connection to a Data Provider interface can be established, the Data Consumer may search for existing datasets by making an inquiry at a Broker Service Provider. The Broker Service Provider may provide the required metadata for the Data Consumer interface to connect to a Data Provider. Such meta data may include the ID and/or authentication mechanism of the data provider interface. Alternatively or additionally, the Data Consumer interface may establish a connection to a Data Provider interface (i.e., with or without involving a Broker Service Provider). In cases in which the information to connect with the Data Provider interface is already known to the Data Consumer interface, the Data Consumer interface may request the data (and the corresponding metadata) directly from the Data Provider interface. Like a Data Provider interface, the Data Consumer interface may log the details of a successful (or unsuccessful) data exchange transaction at a Clearing House, use Data Apps to check, enrich, transform, etc. the data received, or use a Service Provider interface to connect to International Data Spaces (if it does not deploy the technical infrastructure for participation itself).

Similar to the Data Owner being the legal entity that has the legal control over its data, the Data User is the legal entity that has the legal right to use the data of a Data Owner as specified by the usage policy. In most cases, the Data User is identical with the Data Consumer. However, there may be scenarios in which these roles are assumed by different participants.

The identity provider may act as an agent. It may include a Certification Authority (managing digital certificates for the participants of the International Data Spaces), a Dynamic Attribute Provisioning Service (DAPS, managing the dynamic attributes of the participants), and a service named Dynamic Trust Monitoring (DTM, for continuous monitoring of the security and behavior of the network). It may be responsible for issuing technical identities to parties that have been approved to become Participants in the International Data Spaces. The Identity Provider may be instructed to issue identities based on approved roles (see above). The Identity Provider may also manage the PKI rollout. There are two separate PKI hierarchies: one for software signatures (Software Signing Root CA) and one for the IDS Connectors (Service Root CA). An entity is assigned with either an end certificate or a sub/root-CA certificate. The Identity Provider may act as an authentication and/or authorization service by incorporating the DAPS.

Each IDS Connector may run different services and communicate with other IDS Connectors. Using the PKI, an IDS Connector protects the persistent storage of its services and the communication with other IDS Connectors. In order to verify PKI signatures (e.g., for authentication, authorization or for Data Apps that were downloaded), the IDS Connector stores the trusted root certificates (Service Root CA and Software Signing Root CA) in a way their integrity is preserved.

App stores may provide data apps which can be run inside isolated containers of the IDS connectors. These are applications that can be deployed in IDS Connectors to execute tasks like transformation, aggregation or analytics on the data. App stores can be provided by IDS members and must be separately certified under IDS standards. An App Store has a service sub CA.

The International Data Spaces Association signs a certificate signing request (CSR) in order to approve Apps or App Stores. The CSR identifies the App Store and makes it possible to sign the service CSRs from the IDS Connectors requesting apps. The IDS Connector creates a key pair for every App it downloads. The private key protects the App's persistent data. When downloading an App from the App Store, the IDS Connector creates a CSR using the public key. The App Store signs the CSR and issues a certificate. The IDS Connector uses this certificate to make sure that the App it is running and is valid.

Vocabulary providers may manage and offer “vocabularies” (including ontologies, reference data models, metadata elements) which can be used to annotate and describe datasets. Vocabulary providers provide these (domain-specific) vocabularies and their references to the IDS Information Model, which is the basis for the description of data sources.

The Broker may be used as an intermediary that stores and manages information about the data sources available in the International Data Spaces. As the role of the Broker is central but non-exclusive, multiple Brokers may be around at the same time (e.g., for different application domains). An organization offering broker services in the International Data Spaces may assume other intermediary roles at the same time (e.g., Clearing House or Identity Provider, see below). The activities of the Broker may include receiving and providing metadata. The Broker must provide an interface for Data Provider interfaces to send their metadata. The metadata may be stored in an internal repository for being queried by Data Consumers in a structured manner. While the core of the metadata model may be specified by the International Data Spaces, a Broker may extend the metadata model to manage additional metadata elements. After the Broker has provided the Data Consumer interface with the metadata about a certain Data Provider interface, its job is done (i.e., it is not involved in the subsequent data exchange process).

The Clearing House may be an intermediary that provides clearing and settlement services for financial and data sharing or exchange transactions. In the International Data Spaces, clearing activities are separated from broker services, since these activities are technically different from maintaining a metadata repository. As already stated above, it might still be possible that the two roles “Clearing House” and “Broker” are assumed by the same organization, as both roles require acting as a trusted intermediary between the Data Provider interface and the Data Consumer interface. The clearing house and the broker may include a distributed ledger to record metadata from data services. The Clearing House logs activities performed in the course of a data exchange. After a data exchange, or parts of it, are completed, both the Data Provider interface and the Data Consumer interface confirm the data transfer by logging the details of the transaction at the Clearing House. Based on this logging information, the transaction may be checked or billed. The logging information can also be used to resolve conflicts (e.g., to clarify whether a data package has been received by the Data Consumer or not). The Clearing House may provide reports on the performed (logged) transactions for billing, conflict resolution, etc.

FIG. 9 shows a schematic illustration of the authentication process between the IDS connector 1 present in an IDS data provider domain and an IDS connector 2 present in an IDS data consumer domain.

In a typical scenario Connector 2 is associated with a third party that wants to access to chemical product data associated with connector 1 of an chemical product data owner. To provide data from the IDS connector 1 in the IDS data provider domain to the IDS connector 2 present in an IDS data consumer domain the following authentication protocol may be applied.

Connectors are associated with a certificate issued by the Device-CA. This certificate may serve as the root of identity. The connector may include multiple authentication mechanisms or identity tokens, e.g. a device certificate (X.509v3), a TLS connection certificate (X.509v3) and a ‘Dynamic Attribute Token’ (OAuth Access Token).

When data within the data service of IDS connector 1 may be accessed by IDS connector 2, an access token may be presented by IDS connector 2. For this purpose, IDS connector 2 may present its X.509 Device Certificate to the DAPS (Dynamic attribute provisioning service) located in the IDS infrastructure to receive a Dynamic Attribute Token (DAT). The ‘Dynamic Attribute Token’ may include an OAuth Access Token, signed by the Dynamic Attribute Provisioning Service (DAPS). The DAT may be implemented as JSON Web Tokens (JWT) including the cryptographic signature of the DAPS and/or an indication of verified or non-verified identity attributes of connector 2. Dynamic in this context refers to a short-lived token that contains attributes that the IDS connector 2 possesses. Such short-lived token may only be valid for a limited number of transactions, e.g. one transaction. The DAPS may verify the Device Certificate with the Device Sub-CA prior to issuing the DAT.

The following steps may be performed by Connector 2 to receive the DAT:
A: call token endpoint DAPS with Client Credentials (X.509 Cert)
B: Issue JWT-1{attribute_list, client_id, aud: idsAS:*}

After receiving the DAT, a TLS tunnel may be established by requesting a TLS connection certificate using the same X.509 Device Certificate. This certificate may be automatically requested by the IDS connector 2 by interacting with a ACME server that may be integrated into the TLS Sub-CA present in the IDS infrastructure (not shown).

In one example, an Identity Token may be requested by Connector 2 using the authorization service of Connector 1. This step may be optional. The Identity Token may be requested, for example, if several Access Tokens (Ats) are used. For requesting the Identity Token, Connector 2 hands in the DAT at the authorization service of Connector 1 and requests an Identity Token. The authorization service in IDS connector 1 uses a database or rule engine to decide whether the Identity Token is to be provided to Connector 2.

The following steps may be performed by connector 2:
Hand in JWT-1 at connector 1 authentication service,
request JWT-2 {scope: C1/PS}
Use Rule Engine for access decision and issue JWT-2{aud: C1} at connector 1
Provide JWT-2 to connector 2

Connector 1 may provide the Identity Token to Connector 2. Connector 2 hands in the Identity token and optionally DAT to request access to the data within the data service of IDS connector 1 via IDSCP (IDS communication protocol). If no Identity Token needs to be requested, Connector 2 hands in the DAT to Connector 1 to request access to the data within data service of Connector 1 via IDSCP (IDS communication protocol).

Connector 1 may verify the DAT and provide access to the data.

FIG. 10 shows an example implementation of an authorization mechanism using usage-controlled data flow. Such example may be implemented in the international data space (IDS) framework described in FIG. 8.

After access to the IDS connector associated with the data providing service has been granted according to the procedure described in relation to FIG. 9, authorization based on authorization rule(s) may follow. By way of authentication the decentral identifier or data related to the decentral identifier of the accessing entity is available to connector IDS connector.

Usage controlled data flows are one way to enforce authorization rule(s) or usage policies on data exchange or sharing. Usage control may be achieved by binding usage policies to data being exchanged and by continuously controlling the way how data is processed, aggregated or forwarded to other endpoints.

For enforcing usage policies, data flows may be monitored and potentially intercepted by control points. The intercepted data flows may be passed to a decision engine for requesting permission, denial or modification of the data flow. The decision-making may be based on the evaluation of the usage policy by the decision engine. Data usage transactions may trigger interception and evaluation.

To provide the set of authorization rules for the chemical product data associated with the decentral identifier of the IDS connector, usage policies may be provided by a policy manager to the decision engine. The usage policies may be stored independently from the data. The policy manager may be present within the IDS connectors and/or within the clearing house to administer usage policies.

Based on the decentral identifier or data related to the decentral identifier of the accessing entity, the authorization rule for chemical product data associated with a decentral identifier may be selected for enforcement. Enforcement mechanisms may work differently (e.g., work on different system actions) on different systems or technologies. Abstract policies can have different instantiations. Usage policies may be instantiated on the target system. In another example (not shown), usage policies may adhered to the data (also called sticky policy). Sticky policies are one way to cope with the distribution of the usage restrictions. In this approach, machine-readable usage policies may stick to data when it is exchanged. There exist different realization possibilities. For instance, data may be encrypted and can only be decrypted when the adherence to the usage restrictions are guaranteed.

Usage policies may include additional information provided e.g. by a policy information registry. Additional information may include information about contextual information such as previous data usages or the geographical location of an entity, pre- or post-conditions that have to hold before (e.g. integrity checks) and after (e.g. data item is deleted after usage) the decision-making and on-conditions that have to hold during usage (e.g. only during business hours). In case the decision depends on additional information, the decision engine or the control point may request additional information from the policy information registry. The policy information registry information may provide the requested additional information. In addition, the policy information registry may be used to get contextual information for or about the intercepted system action (e.g., data flow information, geolocation of the requesting device). For example, the policy information registry may be used to resolve the ID of a supplier to a postal address and the postal address to GPS coordinates.

The policy executor may be used to perform additional actions based on policy rules, such as sending a notification e.g. via email when data is used or writing to a system log. The action may be triggered by sending the instruction related to the desired additional action from the control point to the policy executor e.g. after the control point receives the additional information from the policy information system. The policy executor may perform the instructed action and may confirm that the action succeeded to the decision engine.

After the decision engine receives the confirmation from the policy executor, it may forward its decision to the requesting control point. The decision can comprise allowing the data flow, denying the data flow or modifying the data flow. The decision may be implemented by the control point. The selected authorization rule may be applied to the chemical product data associated with a decentral identifier. The chemical product data associated with the decentral identifier according to the selected authorization rule may be provided.

The usage control enforcement can be implemented in the IDS connectors. At runtime, the usage control enforcement prevents IDS connectors from treating data in an undesired way, for example by forwarding personal data to public endpoints. Application of the usage policy associated with the usage enforcement control at the data provider connector or at the data consumer connector may depends on the usage policy. At the data provider connector, usage policies may specify how often data can be accessed, at what times (e.g., only within business hours), or that data must be filtered or masked (e.g., anonymized) before leaving the company. The usage policies at the data provider connector may include obligations for the data consumer. The technical enforcement may be handled by the control point or the policy executor depending on the usage restriction. For example, limiting data flowing to a specific target system to ensure the correct usage purpose may be handled by the control point while the deletion of data in storage infrastructure outside the connector may be handled by the policy executor. The usage control enforcement may be implemented at the storage infrastructure. Storage infrastructure may be any kind of storage to persist data such as a file system or a database. The storage infrastructure may be used without modification but every usage of the data may be handled by the IDS connector. In certain instances this could lead to a bottleneck. Usage control may be implemented by encrypting the data within the IDS connector connected to the storage infrastructure before transferring the data to the storage infrastructure. Using the data is only possible by using the IDS connector to decrypt the data. Hence, every usage is controlled by the IDS connector. In such cases, usage restrictions such as data lifetime or time constraints can be enforced by deleting the cryptographic key material. Additionally or alternatively, the storage infrastructure may include a usage control enforcement component that monitors and/or controls the usage of the data.

Usage control enforcement may also be implemented in applications such that the data flow within the applications can be controlled and adhered to the usage policies. Similar to the storage infrastructure, a control point can be integrated into the application that controls the data flows.

The afore-described usage control enforcement in the IDS allows to achieve the following security requirement, which cannot be achieved using traditional access control:
Secrecy: Classified data must not be forwarded to nodes which do not have the respective clearance,
Integrity: Critical data must not be modified by untrusted nodes as otherwise their integrity cannot be guaranteed anymore,
Time to live: A prerequisite for persisting data is that it must be deleted from storage after a given period of time,
Anonymization by aggregation: Personal data must only be used as aggregates by untrusted parties. A sufficient number of distinct records must be aggregated in order to prevent deanonymization of individual records,
Anonymization by replacement: Data which allows a personal identification (e.g. faces in camera images) must be replaced by an adequate substitute (e.g. blurred) in order to guarantee that individuals cannot be deanonymized from the data,
Separation of duty: Two data sets from competitive entities (e.g. two chemical industries) must never be aggregated or processed by the same service,
Usage scope: Data may only serve as input for data pipes within the connector but must never leave the connector to an external endpoint.

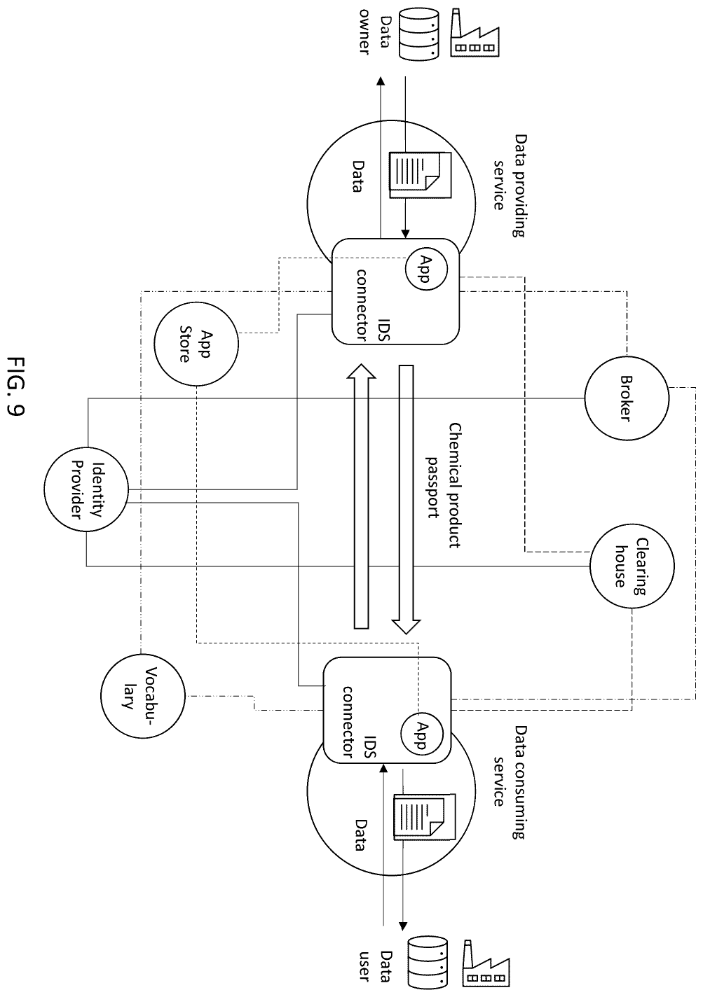

FIG. 12 illustrates an example of an apparatus and associated methods for generating the product passport in connection with a chemical product produced by a chemical production network.

The chemical production network 1000 may produce at least one chemical product 1004 from one or more raw material(s) 1002. The raw material(s) 1002 may enter the system boundary 1001 of the chemical production network 1000. The chemical product 1004 may be produced using the raw material(s) 1002. The chemical product 1004 may exit the system boundary 1001 of the chemical production network 1000.

Upon producing the chemical product 1004 or exiting of the chemical product 1004 of the chemical production network 1000, the chemical product passport may be generated. The apparatus 1010 may be configured to generate the chemical product passport. A requestor 1008 may be configured to provide the decentral identifier. The requestor may be configured generate the request for the decentral identifier. Such request may be triggered by a labelling system such as a QR Code generator. The request to provide the decentral identifier may be provided to a decentral identifier generator 1012 configured to generate the decentral identifier. The decentral identifier generator 1012 may provide the decentral identifier to a decentral identifier provider 1014. The decentral identifier provider 1014 may provide the decentral identifier to the requestor 1008 configured to associate the decentral identifier with the chemical product. Such association may include encoding the decentral identifier into a QR code and providing the QR code for labelling the chemical product. This way a physical identifier may be provided that relates the physical entity of the chemical product with the decentral identifier.

The decentral identifier generator 1012 may provide the decentral identifier to a passport generator 1016 configured to generate the chemical product passport. The passport generator 1016 may generate the chemical product passport as described for example in the context of FIGS. 2-4. The chemical product passport may include the decentral identifier and data related to the chemical product data. The data related to chemical product data may include one or more digital representation(s) pointing to chemical product data or parts thereof. The chemical product passport may include or be related to one or more authentication mechanisms associated with the decentral identifier and the data related to the chemical product data. The authentication mechanisms may be used as described for example in the context of FIGS. 5-11. The chemical product passport may relate to one or more authorization mechanisms associated with the decentral identifier and the data related to chemical product data. The authorization mechanisms may be used as described for example in the context of FIGS. 5-11.

The chemical product passport may be provided to a chemical product passport provider 1018. The chemical product passport provider 10018 may be configured to provide the chemical product passport for access by a data consuming service 1020 under control by a data providing service associated with the data owner. The chemical product passport may be used to access chemical product data as for example described in the context of FIGS. 5-11.

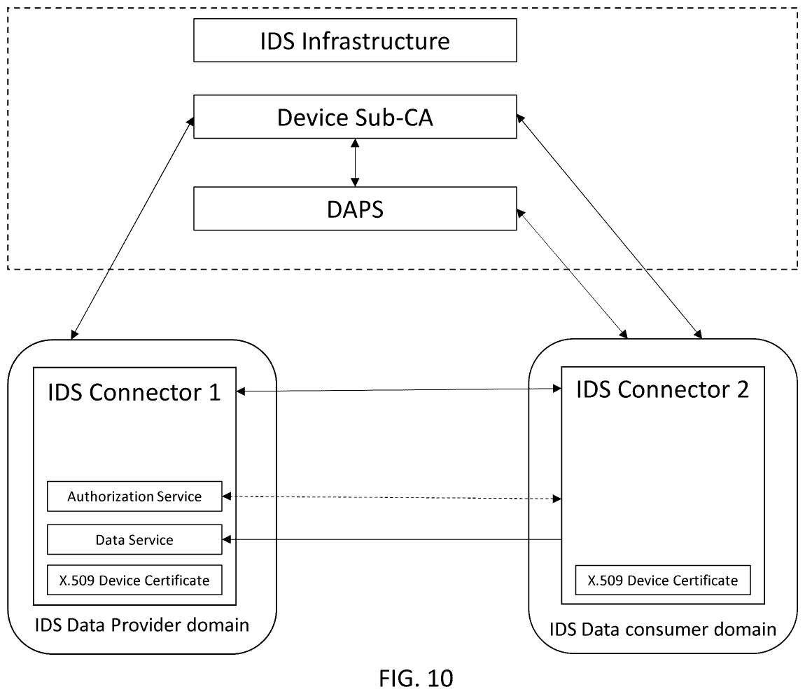

FIGS. 13-18 show different example configurations for product passports anchored by digital identifiers. The configurations include different parent, child, grandchild and so on relationships for product passports generated in the chemical value chain up to an end product.

FIG. 13 illustrates an individual configuration for different product passports generated in the chemical value chain. For multiple product stages in the chemical value chain individual product passports may be generated. The product passport generation may include the providing of a decentral identifier and an authentication mechanism for each of the multiple product stages. The product passports for the multiple product stages may be based on crypto signatures. For instance, the product passports for the multiple product stages may be concatenated through hash values based on different data sets. As shown in FIG. 12 hash 1 may be based on data of the raw material passport, hash 2 may be based on data of the chemical product passport and hash 3 may be based on data of the raw material passport plus data of the chemical product passport. Further concatenation may be done for other combinations of product passports up to hash n, which concatenates product passports up to the end product passport. Concatenation via hashes of the crypto signature is only one example concatenation. Other examples include permission aggregations with different scope of data that may be embedded in child passports, public key aggregations with different crypto signatures or service endpoint aggregation with different links.

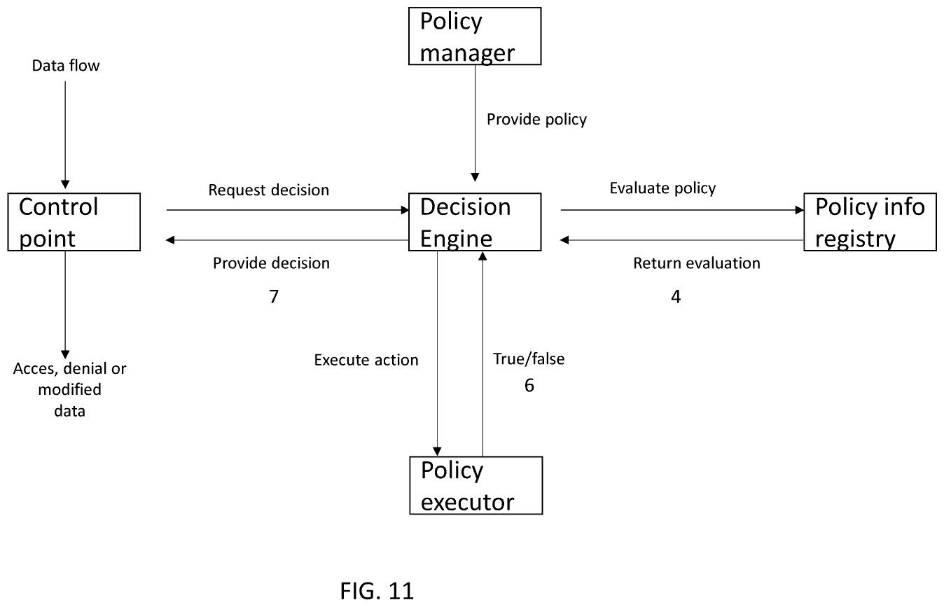

FIG. 14 illustrates different product passports including a concatenation associated with multiple decentral identifiers based on the relationship representation for different products associated with product stages of the chemical value chain. In this particular example hash values are used for the concatenation associated with multiple decentral identifiers. The data sets to generate the hash values are schematically illustrated in FIG. 13.

The raw material passport may be provided to the chemical product producer using the raw material to produce the chemical product. The raw material passport may be connected to a hash value hash 1. The hash value hash 1 may be generated via a hashing algorithm such as MD5, SHA-1, SHA-2, SHA-3 or any other suitable algorithm based on a one-way function that can't be reverse engineered. The hash value hash 1 may be generated based on data included in or connected to the raw material passport. The data for hash generation may include the decentral identifier and the data related to the raw material data. The data for hash generation may include the raw material data. The data for hash generation may include the decentral identifier associated with the raw material, the data related to the raw material data, the raw material data associated with the raw material and/or cryptographic information connected to the decentral identifier. The hash value hash 1 may be used by participant nodes of the chemical supply chain to check integrity of the data package transferred from the raw material supplier e.g. to the chemical product producer.

Similar to the raw material passport, the chemical product passport may be provided to the component producer using the chemical product to produce the component. The generation of the chemical product passport may be based on the raw material passport provided to the chemical product producer using the raw material to produce the chemical product. The chemical product passport may be connected to one or more hash value(s) hash 2, hash 3. The hash values hash 2, hash 3 may be generated via a hashing algorithm such as MD5, SHA-1, SHA-2, SHA-3 or any other suitable algorithm based on a one-way function that can't be reverse engineered. The hash values hash 2, hash 3 may be generated based on data included in or connected to the chemical product passport and/or the raw material passport. The hash value(s) hash 2, hash 3 may be generated based on clear data itself or based on hash value(s) generated from the clear data. For instance, hash 3 may be generated based on passport data or based on hashed passport data. The data for hash generation may include the decentral identifier associated with the raw material used to produce he chemical product, the decentral identifier associated with the chemical product, the data related to the raw material data and/or the data related to the chemical product data.

The relationship representation may include material flow or product information, such that the concatenation may relate to the decentral identifiers associated with the individual product stages, such as chemical product and the raw material(s). Hashing data related or included in the respective passports may provide for such concatenation. The data for hash generation may include the raw material data and/or the chemical product data. The data for hash generation may include the decentral identifier associated with the raw material, the data related to the raw material data, the raw material data associated with the raw material and/or cryptographic information connected to the decentral identifier. The data for hash generation may include the decentral identifier associated with the chemical product, the data related to the chemical product data, the chemical product data associated with the chemical product and/or cryptographic information connected to the decentral identifier. Hash value hash 2 may be generated in relation to the chemical product passport as illustrated in FIG. 13. The hash value hash 2 may be based on at least the decentral identifier associated with the chemical product and the data related to the chemical product data. Hash value hash 3 may be generated in relation to the raw material passport and the chemical product passport as illustrated in FIG. 13. The hash value hash 3 may be based on at least the decentral identifier associated with the raw material, the data related to the raw material data, the decentral identifier associated with the chemical product and the data related to the chemical product data. The hash value(s) may be used by participant nodes of the chemical supply chain to check integrity of the data package transferred from the raw material supplier or the chemical product producer. The combined hash value(s) may further be used by participant nodes of the chemical supply chain to determine the relation of products at different stages and to check integrity of such relation.

As illustrated in FIGS. 13 and 14, the hash value(s) may be connected to the product passports associated with one or more product stage(s) of the chemical supply chain.

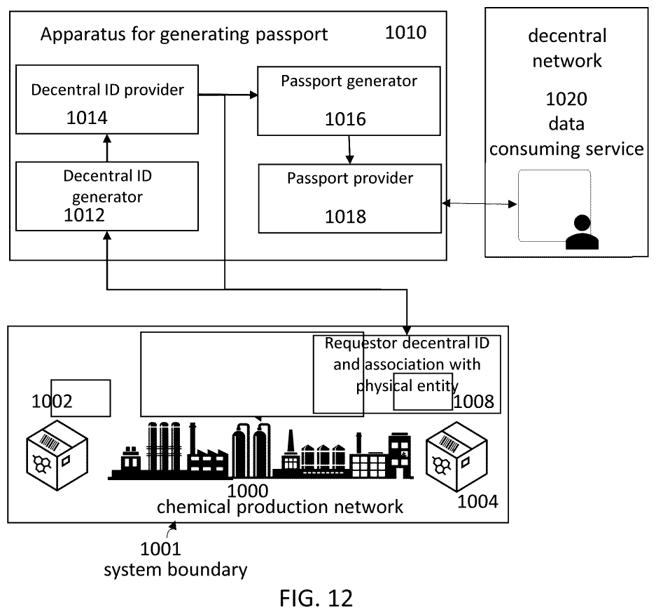

FIG. 15 illustrates an anchored configuration for different product passports generated in the chemical value chain. For the end product an end product passport is generated. For multiple further product stages in the chemical value chain individual product passports may be generated and embedded in or linked with the end product passport. The product passport generation may include the providing of a decentral identifier and an authentication mechanism for each of the multiple product stages. The product passports for the multiple product stages may be based on crypto signatures. For instance, the product passports for the multiple further product stages may be concatenated through hash values based on different data sets. As shown in FIG. 13 hash 1 may be based on data of the raw material passport, hash 2 may be based on data of the chemical product passport and hash 3 may be based on data of the raw material passport plus data of the chemical product passport. Further concatenation may be done for other combinations of product passports up to hash n, which concatenates product passports up to the end product passport. Concatenation via hashes of the crypto signature is only one example concatenation. Other examples include permission aggregations with different scope of data that may be embedded in child passports, public key aggregations with different crypto signatures or service endpoint aggregation with different links.

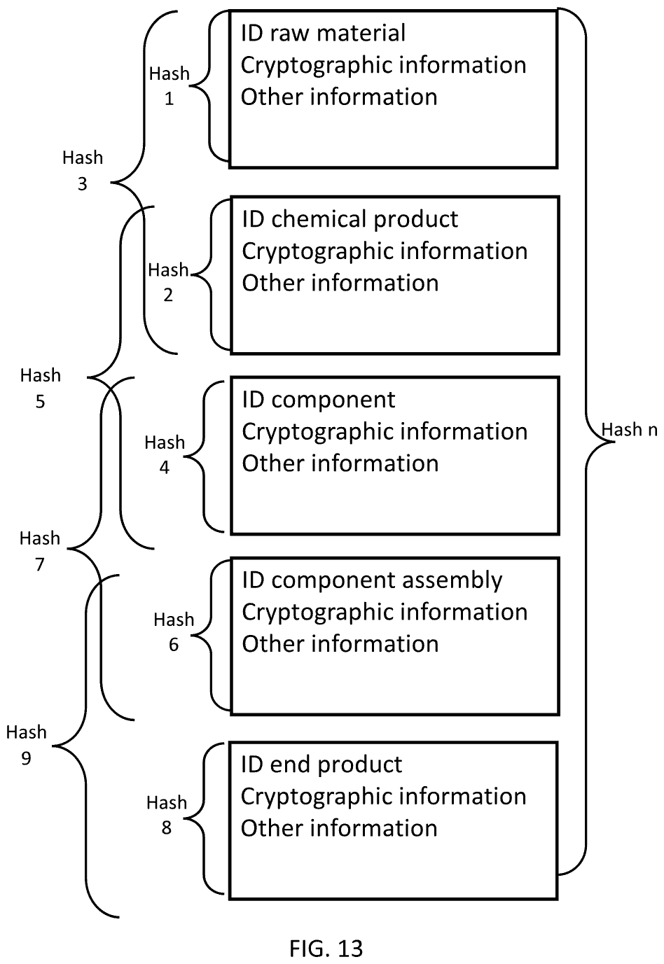

FIG. 16 illustrates different product passports including a concatenation associated with multiple decentral identifiers based on the relationship representation for different products associated with product stages of the chemical value chain. In this particular example hash values are used for the concatenation associated with multiple decentral identifiers. The data sets to generate the hash values are schematically illustrated in FIG. 15.

The raw material passport may be provided to the chemical product producer using the raw material to produce the chemical product. The raw material passport may be connected to a hash value hash 1 that may be generated as described in the context of FIG. 14.

Similar to the raw material passport, the chemical product passport may be provided to the component producer using the chemical product to produce the component. The hash values may be generated as described in the context of FIG. 15. The hash values may be generated in a similar fashion for the product passports up to the end product passport.

In the anchored configuration, the end product passport may include hash values that relate to the product passports associated with the products used to produce the end product. The relationship representation may include material flow or product information, such that the concatenation may relate to the decentral identifiers associated with the individual product stages, such as chemical product and the raw material(s). The concatenation may in this case relate to the decentral identifiers associated with the chemical product(s), the raw material(s), the component(s) and the component assembly/ies. Hashing data related or included in the respective passports may provide for the concatenation. The data for hash generation may include any data included in respective passports. The hash value hash 7 may be based on at least the decentral identifier associated with the end product and the data related to the end product data. Hash value hash 8 may be generated in relation to the product passports associated with products at different product stages as illustrated in FIG. 15. The combined hash value hash 8 may be used by participant nodes of the chemical supply chain to determine the relation of products at different stages and to check integrity of such relation.

As illustrated in FIGS. 15 and 16, the hash value(s) may be connected to the product passports associated with one or more product stage(s) of the chemical supply chain.

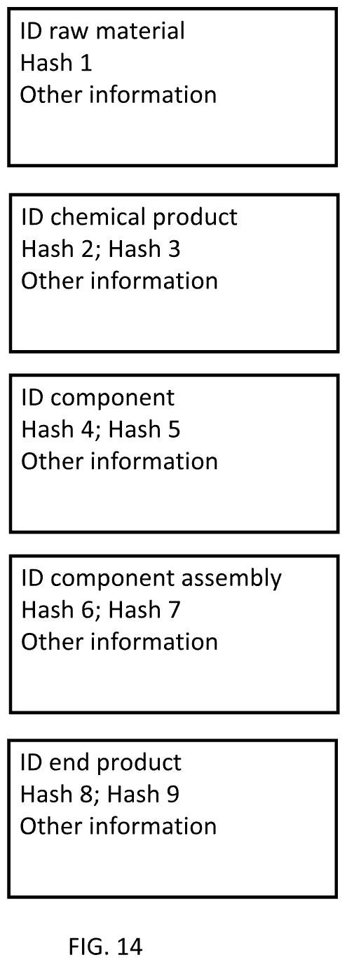

FIG. 17 illustrates a fully embedded configuration for different product passports generated in the chemical value chain. For multiple product stages in the chemical value chain individual product passports may be generated. The product passport generation may include the providing of a decentral identifier and an authentication mechanism for each of the multiple product stages. The product passports for the multiple product stages may be based on crypto signatures. For instance, the product passports for the multiple product stages may be concatenated through hash values based on different data sets. As shown in FIG. 14 hash 1 may be based on data of the raw material passport. Hash 2 may be based on data of the raw material passport and the chemical product passport. Further concatenation may be done for other combinations of product passports up to hash n, which concatenates product passports up to the end product passport. Concatenation via hashes of the crypto signature is only one example concatenation. Other examples include permission aggregations with different scope of data that may be embedded in child passports, public key aggregations with different crypto signatures or service endpoint aggregation with different links.

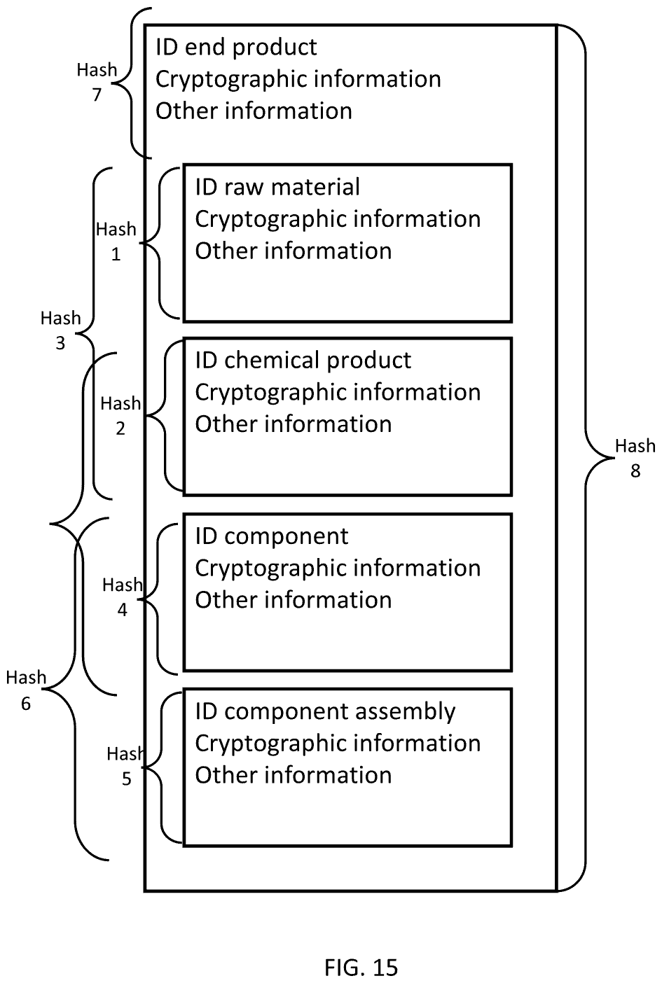

FIG. 18 illustrates different product passports including a concatenation associated with multiple decentral identifiers based on the relationship representation for different products associated with product stages of the chemical value chain. In this particular example hash values are used for the concatenation associated with multiple decentral identifiers. The data sets to generate the hash values are schematically illustrated in FIG. 17.

The raw material passport may be provided to the chemical product producer using the raw material to produce the chemical product. The raw material passport may be connected to a hash value hash 1 that may be generated as described in the context of FIG. 14. Similar to the raw material passport, the chemical product passport may be provided to the component producer using the chemical product to produce the component. The hash values may be generated as described in the context of FIG. 17. The hash values may be generated in a similar fashion for the product passports up to the end product passport.

In the fully embedded configuration, the combined hash values may be generated from product passports associated with all products preceding the product. The relationship representation may include material flow or product information, such that the concatenation may relate to the decentral identifiers associated with the individual product stages, such as chemical product and the raw material(s). The concatenation may in this case relate to the decentral identifiers associated with all preceding products, such as the chemical product(s), the raw material(s), the component(s) and the component assembly/ies. Hashing data related or included in the respective passports may provide for the concatenation. The data for hash generation may include any data included in respective passports. The hash value hash 7 for example may be based on at least the decentral identifier associated with the products up to the end product and the data related to the respective data. Hash value hash 9 for example may be based on at least the decentral identifier associated with the products up to and including the end product and the data related to the respective data. The combined hash values hash, 3, hash 5, hash 7 and hash 9 may be used by participant nodes of the chemical supply chain to determine the relation of products at different stages and to check integrity of such relation. As illustrated in FIGS. 17 and 18, the hash value(s) may be connected to the product passports associated with one or more product stage(s) of the chemical supply chain.

The configurations shown in FIGS. 13-18 relate to product passports generated in the chemical product value chain up to an end product. Similarly, product passports generated in the recycling product chain from end product to recyclate(s) may be concatenated. Further similarly, the product passports generated in the chemical product chain up to an end product and in the recycling chain from end product to recyclate(s) may be concatenated. This way the circularity of products and in particular materials may be virtually represented and tracked.

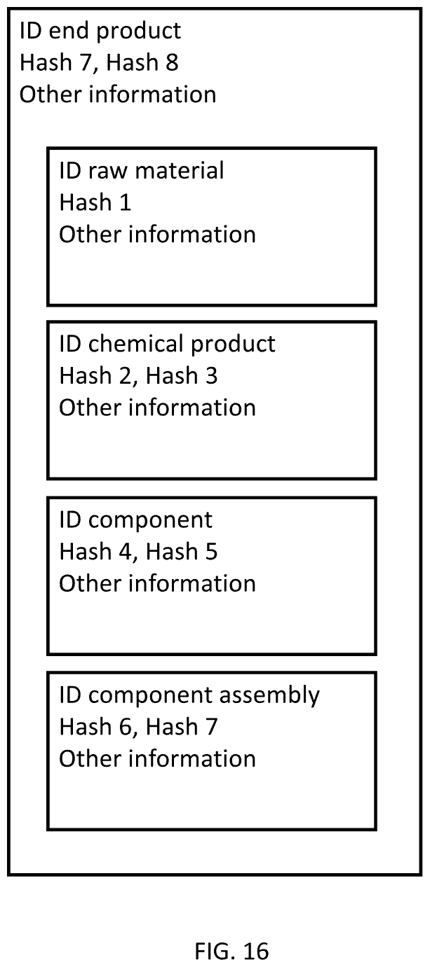

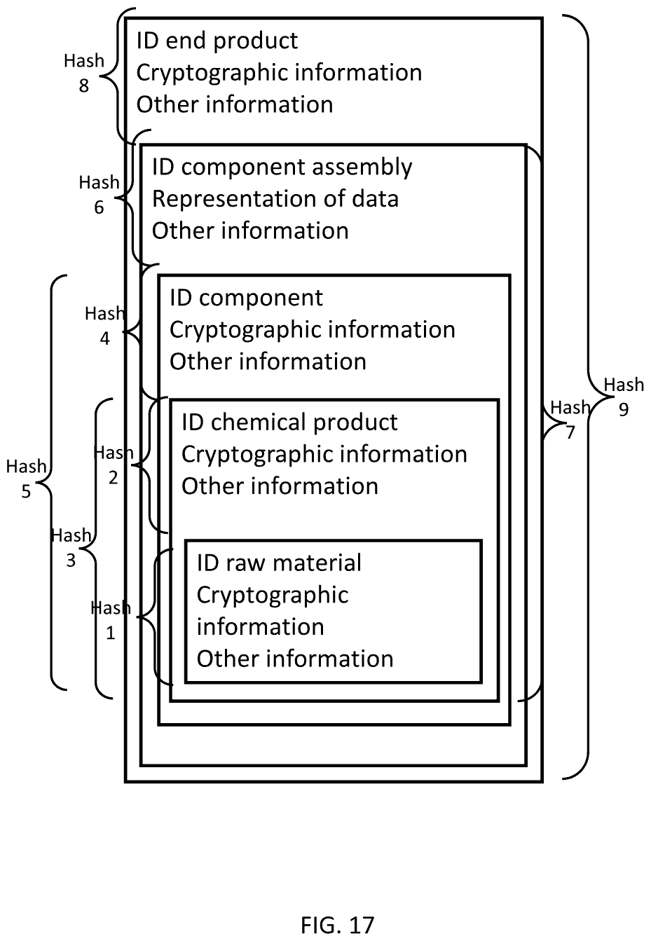

FIGS. 19 and 20 illustrate examples of relationship representations, which may be used for generating the concatenation.

The relationship representation may relate to different stages of the product supply chain and recycling product chain. The product passports at different stages of the product supply chain and recycling product chain may be signified by the relationship representation. The relationship representation may be associated with the product produced at the respective stage of the product supply chain and recycling product chain and at least one product used to produce the respective product. The relationship representation may be associated with the product produced at a respective stage and at least one product produced at a previous stage of the product supply chain and recycling product chain. The relationship representation may specify the relation between physical entities. The relationship representation may specify that the second physical entity may be used to produce the first physical entity as illustrated in FIG. 19 and/or that the first physical entity may be produced by using the second physical entity as illustrated in FIG. 20. The relationship representation may relate to at least one raw material used to produce the chemical product. The relationship representation may relate to at least one end product used to produce the recycling product or recycled product. The relationship representation may relate to the chemical product produced by using at least one raw material.

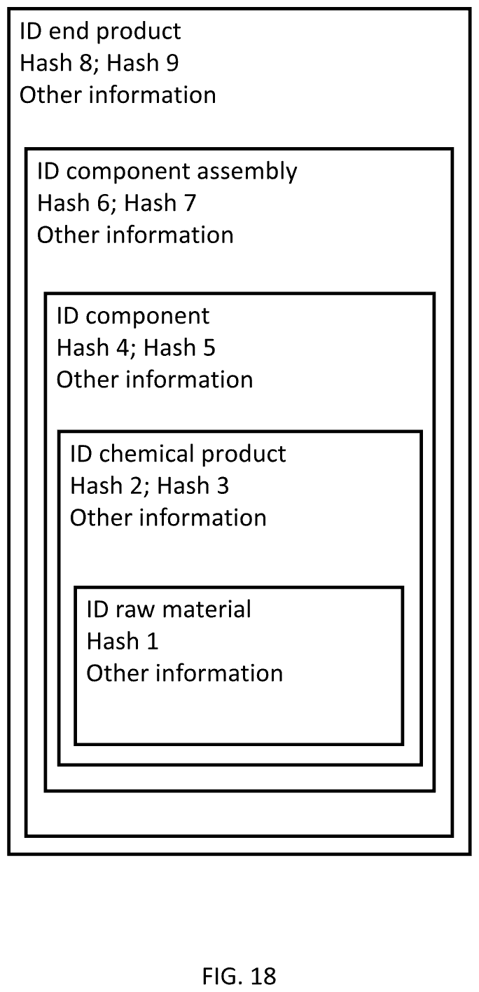

FIG. 21 illustrates an example of an apparatus for generating and using the concatenation in a decentral network. The example may be applicable to any node associated with participants of the product supply chain, such as the chemical product supply chain, and/or the recycling product chain. For illustrative purposes the example will be explained for the node associated with the chemical product producer and the node associated with the component producer using the chemical product to produce the component. The concatenation may be generated on generation of the component passport. The concatenation may be generated separately from generation of the component passport.

For generation of the concatenation, the node associated with the component producer may receive the chemical product passport. The chemical product passport may include or be connected to the decentral identifier associated with the chemical product and the data related to the chemical product data as described in the context of FIGS. 2-4.

The node associated with the component producer may access the chemical product data as described in the context of FIGS. 5-11. The node associated with the component producer may access the chemical product data. The node associated with the component producer may generate or provide a relationship presentation which signifies the relation of the physical entities of the product(s).

The node associated with the component producer may generate the component passport. The node associated with the component producer may generate the concatenation based on the relationship representation. The node associated with the component producer may generate the concatenation based on the decentral identifier associated with the component, the data related to the component data and/or the component data as well as the decentral identifier associated with the chemical product, the data related to the chemical product data and/or the chemical product data. All variants described in the context of FIGS. 13-18 may be applicable

The concatenation may be provided by the component producer via the component passport associated with the physical entity of the produced component and/or to at least one authentication registry. The authentication registry may store the concatenation associated with the decentral identifiers. On request the authentication registry may resolve the concatenation to track and trace physical entities of the product chain. In addition or alternatively, any node may request resolution of the concatenation generated based on data available to the nodes. Based on such relationship presentation, the nodes having access to the data required to generate the hash may resolve the concatenation to track and trace physical entities of the product chain.

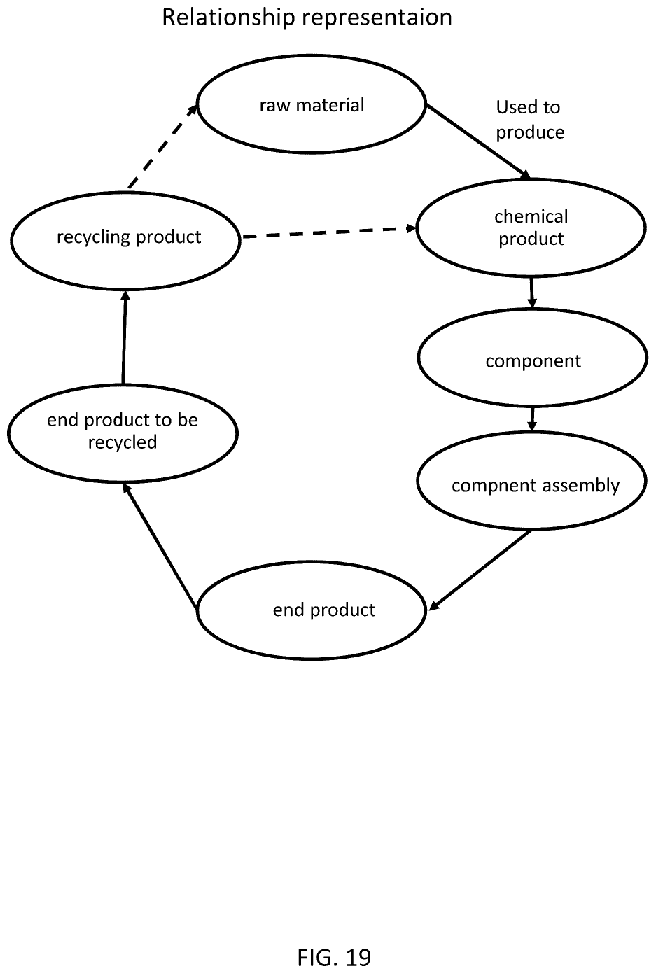

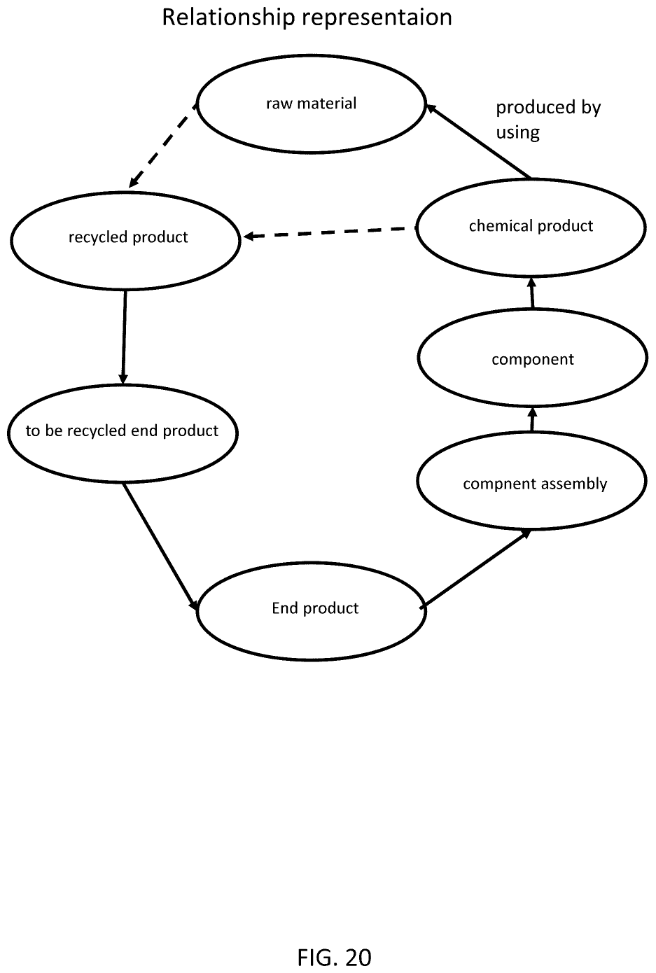

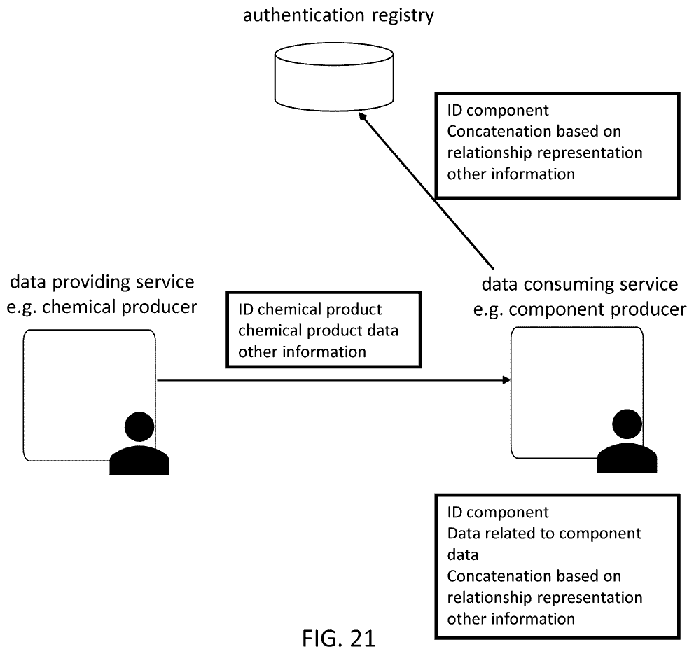

FIG. 22 illustrates one example of a production facility producing a chemical product associated with a chemical product passport.

The production facility illustrated in FIG. 22 may manufacture the chemical product. The production facility may manufacture, e.g., an organic chemical product obtained by reacting organic chemical reactants. The production facility may include one or more production plants. For example, the production facility for manufacturing the chemical product may include at least one precursor/intermediate product production plant. The production plant may use physical inputs representing one physical entity used to produce an output product. The production plant may produce physical outputs representing another physical entity in the form of the produced product.

The physical inputs to the production facility may include materials, such raw materials, intermediate materials or components to be assembled. Raw materials may be virgin or recycled raw materials.

The physical inputs may be associated with the decentral identifier as described above. The physical input(s) may be registered with the production facility. The registration may include providing the decentral identifier associated with the physical inputs. Providing the decentral identifier may include reading the physical identifier element physically connected to the physical input as described above. Providing the decentral identifier may include accessing a data base with decentral identifiers and fetching the decentral identifier associated with the physical input.

Based on the provided decentral identifier, chemical product data associated with such decentral identifier may be accessed. Access may be granted through authentication and authorization based on authentication and authorization information associated with the decentral identifier. Based on the decentral identifier, chemical product data such as chemical product declaration data, chemical product safety data, certificate of analysis data, emission data, product carbon footprint data, product environmental footprint data, chemical product specification data, product information, technical application data, production data, performance data, quality data, material configuration data, recyclate content data or combinations thereof may be accessed. Chemical product data may be accessed through a data service requesting access to the chemical product data associated with each decentral identifier and controlled by a physical input data owner. Data owner may be the producer of the physical inputs. The data service may include computer executable instructions operating in an at least partially decentral computing environment. Such computer executable instructions may be based on a Json Web Token (JWT) including authentication information, authorization information and/or a digital representation pointing to chemical product data or parts thereof. The digital representation may include an endpoint for data exchange or sharing (resource endpoint) or an endpoint for service interaction (service endpoint), that may be uniquely identified via a communication protocol. The digital representation(s) pointing to chemical product data or parts thereof may be uniquely associated with the decentral identifier. Chemical product data may be used for the manufacturing process of the production facility.

The production facility may produce the physical output based on one or more physical input(s). The physical output of the production facility may be associated with a physical identifier. The physical output of the production facility may be physically connected to a physical identifier element as described above. The physical identifier may be assigned to identifier information associated with the decentral identifier. For such assignment, the physical identifier element may be read or a data base with physical identifiers may be accessed. A request to provide a decentral identifier may be triggered to assign the physical identifier to the decentral identifier. This way, the decentral identifier may be assigned to the physical output. Providing the decentral identifier may include accessing a data base with decentral identifiers and associated information such as authentication information. Providing the decentral identifier may include accessing a decentral service to provide the decentral identifier and associated information such as authentication information. In response to the request, the chemical product passport including the decentral identifier and data related to the chemical product data of the chemical product may be generated. The data related to the chemical product data may include a representation such as a pointer or a link to the chemical product data. The decentral identifier may be associated to data related to the chemical product data of the physical output. The decentral identifier may further be related or assigned or linked to the decentral identifier of the physical input as lined out in FIGS. 13 to 21. The chemical product passport may be provided to a decentral network for access by other participants or producers of the network. This way the chain of input to output material may be made traceable and usable in further manufacturing steps without exposing the chemical product data in an uncontrolled manner.

The process steps described above may be executed via an operating system of the production facility In this embodiment, the operating system includes a collector configured to collect chemical product data and/or physical identifiers as described above. The collector may be configured to collect chemical product data associated with the chemical product or chemical product data associated with the production of the chemical product, wherein the chemical product is connected to or comprises the physical identifier. The operating system may in particular include an ID reader configured to provide the physical identity of physical input(s) or output(s) as described above. The system may further include an assignor configured to assign the decentral identifier and associated information to the physical output as described above.

Further the operating system may include an ID provider configured to provide the decentral identifier and associated information as described above.

FIG. 16 illustrates another example of a production facility producing a chemical product associated with a chemical product passport.

The process steps described in the context of FIG. 15 may be executed via an operating system of the production facility in interaction with the assignor, the collector or reader or the ID provider. In this embodiment, the operating system may be communicatively connected to the production facility and the assignor, the collector, the ID reader or the ID provider. The operating system may be configured to provide chemical product data from the production facility. The operating system may include a collector configured to provide chemical product data from the production facility. The operating system may include the ID reader configured to read the physical identifier element physically connected to the physical input or output. The assignor may be configured to assign the decentral identifier and associated information to the physical identifier of the physical output as described above in the context of FIG. 15. The ID provider may be configured to provide the decentral identifier and associated information as described above in the context of FIG. 15. The ID reader may be configured to provide the physical identity and associated information of physical input(s) or output(s) as described above in the context of FIG. 15. The assignor, the collector, the ID reader and/or the ID provider may be configured as decentral services or applications executed via the decentral network.

FIGS. 15 and 16 only show two example embodiments and any combination of the system components shown in FIGS. 15 and 16 may be possible. For instance, the ID reader may be configured as part of the operating system, while ID provider and assignor may not be configured as part of the operating system.

FIG. 17 illustrates an example for tracking material in the production of a chemical product from raw materials to the end product. FIG. 17 specifically illustrates an example for tracking material in the production of a chemical product.

For producing a chemical product, raw materials may be provided as physical inputs representing one possible physical entity. The raw materials may comprise precursor materials. The raw materials may include virgin or recycled materials. The raw materials may be associated with a decentral identifier. The decentral identifier may be associated with a digital twin of the raw materials. The decentral identifier may be associated with raw materials data such as a tag for virgin or recyclate material or material properties related to their environmental impact.

The production of the chemical product may comprise a two-step process: 1) production of precursor material, 2) production of the chemical product. To produce the precursor material, the raw materials may be used as physical inputs. The operating system of the precursor production may access data related to the raw materials based on the decentral identifier e.g. from a raw materials provider. Such data may be used to operate the production. For instance, if the raw materials are recycled materials, production steps purifying the recyclate may be comprised. For instance, if the raw materials are virgin materials, purification steps may be omitted. The precursor material may be formed by reactions of the raw materials. Production data from precursor production may be stored and/or associated with a decentral identifier.

In a second step, the precursor material may be provided to produce the chemical product. The precursor material may comprise the precursor produced by the precursor production. The precursor material may comprise recycled precursor material or precursor material produced by a different entity. Such precursor material may be associated with a decentral identifier via which data related to the precursor material may be accessible.

The produced and packaged chemical product may be assigned to a decentral identifier and associated information as lined out above. The packaged chemical product may, for instance, comprise the physical identifier element, such as a QR-Code, physically attached to the package. Such physical identifier element may be assigned to the decentral identifier. The assignment of physical identifier element and decentral identifier may be executed through an ID generator/assignor running locally, in a decentral system and/or in a distributed system.

For instance, the packaging line may comprise a detector detecting the physical identifier on each package. Based on such recognition the operating system of the chemical product production may request to provide a decentral identifier and the provided decentral identifier may be assigned to the physical identifier. In response to the request, the chemical product passport including the decentral identifier and data related to the chemical product may be generated. For such generation, data related to the chemical product as recorded prior and/or during production of the chemical product may be gathered or accessed. Such data may be provided by the operating system of the chemical product production, or a storage environment connect to the operating system of the chemical product production. This may include data collected and stored during precursor material production.

The data related to the chemical product may include the identifier of raw materials used to produce the chemical product. The data related to the chemical product may include the data related to the raw material as e.g. accessible via the decentral identifier of the raw materials.

The data related to the chemical product may include data related to the material configuration of the chemical product. The material configuration data may relate to the chemical composition of the chemical product. The material configuration data may specify at least one constituent of the chemical composition of the chemical product.

The data related to the chemical product may include data related to properties related to environmental impact such as CO₂ footprint or recyclate content. For instance, the data related to the chemical product may specify recyclate content for the components or raw materials. Such recyclate content may either be directly associated with the decentral identifier of the chemical product or it may be indirectly associated with the decentral identifier of the chemical product, e.g. via the decentral identifier of the raw materials or the precursor materials. The data related to the chemical product may include data related to the production conditions as provided by the operating system of the chemical product production. The data related to the chemical product may include data related to the operation conditions as provided by the operating system of the chemical product production. The data related to the chemical product may include data related to the producer, such as producer name, producer brand or producer identifier. The data related to the chemical product may include data related to the product, such as product name, product brand or product identifier.

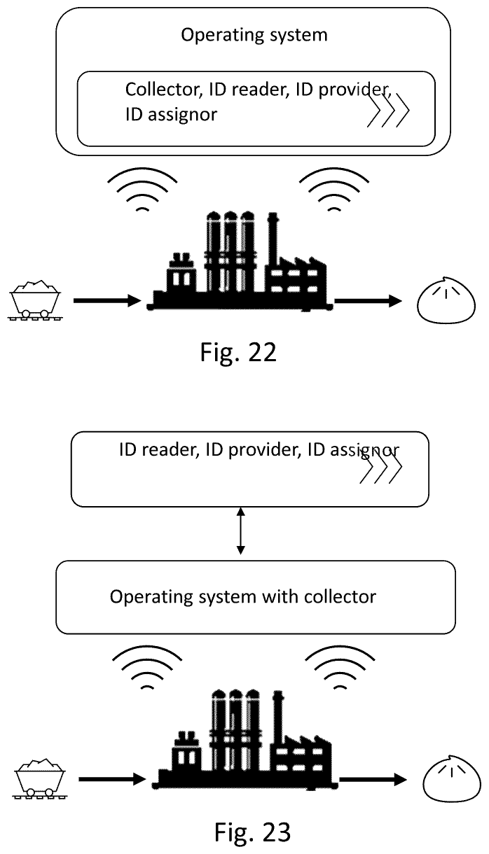

FIGS. 25 to 27 illustrate examples of providing access to data via one or more chemical product passport(s).

Via the decentral setup described above, different supply chain participants or producers up to end consumers may access data from supply chain participants or producers. Access to such data may include transferring the data to the requester or processing the data and transferring the processed result to the requester. The example of FIG. 25 shows data that may be accessed based on the decentral identifier of an end product, e.g. a car. Material data may be provided from different material providers, that have supplied material for the production of the car. Environmental impact data may be provided aggregated from different material providers, that have participated in the production of the car. Lifetime data may be provided as collected during use of the end product.

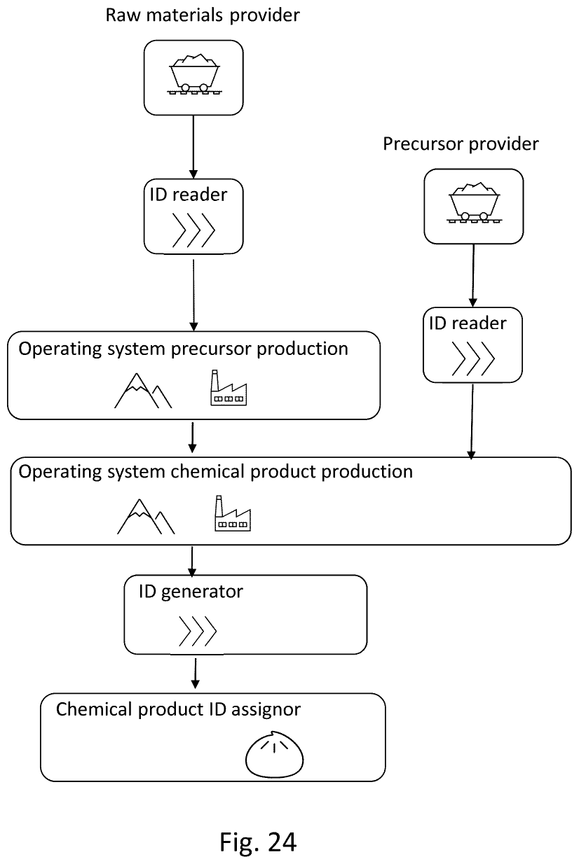

The example of FIG. 26 shows chemical product data as provided by the chemical product producer, precursor A data as provided by the precursor A producer/provider, precursor B data as provided by the precursor B producer/provider.

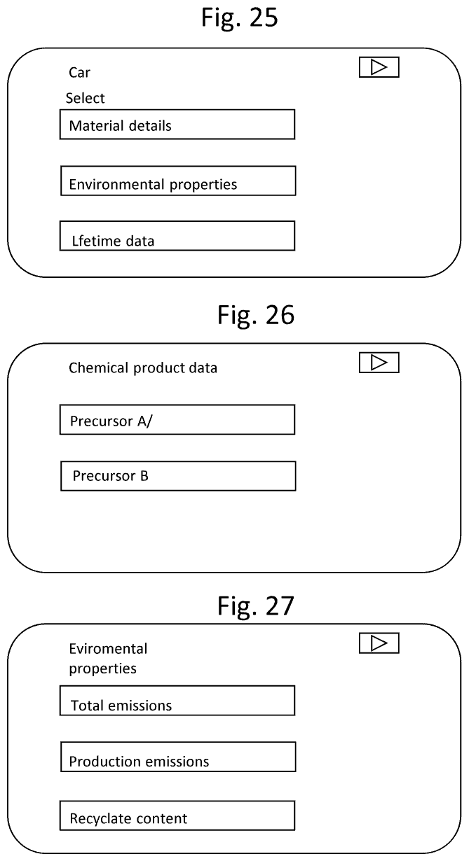

The example of FIG. 27 shows environmental data such as total emissions data as aggregated from data provided by producers of components of the car, production emission data as provided by producers of components of the car, recyclate content data as provided by producers of components of the car.

The present disclosure has been described in conjunction with a preferred embodiment as examples as well. However, other variations can be understood and effected by those persons skilled in the art and practicing the claimed invention, from the studies of the drawings, this disclosure and the claims. Notably, in particular, the any steps presented can be performed in any order, i.e. the present invention is not limited to a specific order of these steps. Moreover, it is also not required that the different steps are performed at a certain place or at one node of a distributed system, i.e. each of the steps may be performed at a different nodes using different equipment/data processing units.

In the claims as well as in the description the word “comprising” does not exclude other elements or steps and the indefinite article “a” or “an” does not exclude a plurality. “can” or “may” refers to optional features. A single element or other unit may fulfill the functions of several entities or items recited in the claims. The mere fact that certain measures are recited in the mutual different dependent claims does not indicate that a combination of these measures cannot be used in an advantageous implementation.

The present disclosure has been described in conjunction with preferred embodiments and examples as well. However, other variations can be understood and effected by those persons skilled in the art and practicing the claimed invention, from the studies of the drawings, this disclosure and the claims.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

All terms and definitions used herein are understood broadly and have their general meaning.

1. An apparatus for generating a product passport, the apparatus comprising: one or more computing node(s) and one or more computer-readable media having thereon computer-executable instructions that are structured such that, when executed by the one or more computing node(s), cause the apparatus to perform the following steps:
receiving a request to provide a first decentral identifier associated with at least one first physical entity of a product supply chain and/or a recycling product chain;
in response to the request, providing the first decentral identifier associated with the at least one first physical entity of the product supply chain and/or the recycling product chain;
generating a product passport including the first decentral identifier and data related to the at least one first physical entity of the product supply chain and/or the recycling product chain; and
providing the product passport for access by a data consuming service under control of a data providing service associated with a data owner.

2. An apparatus for producing a product associated with a product passport, wherein the product comprises at least one first physical entity of a product supply chain and/or a recycling product chain, the apparatus comprising:
at least one collector configured to collect product data associated with at least one first physical entity of the product supply chain and/or the recycling product chain, wherein the at least one first physical entity comprises at least one physical identifier;
at least one assignor configured to assign the physical identifier to a first decentral identifier for generating the product passport associated with the at least one first physical entity; and
a product passport generator configured to generate the product passport by receiving a request to provide at least the first decentral identifier associated with product data of the at least one first physical entity and in response to the request, generating the product passport including the first decentral identifier and data related to the product data of the at least one first physical entity.

3. The apparatus of claim 1, wherein at least one second decentral identifier associated with a second physical entity is provided, wherein the first decentral identifier associated with the first physical entity is connected to the at least one second decentral identifier associated with the second physical entity, wherein based on the first decentral identifier, the at least one second decentral identifier and a relationship representation associated with a relationship between the at least one first physical entity and the at least one second physical entity a concatenation is generated.

4. The apparatus of claim 1, wherein at least one second decentral identifier associated with a second physical entity is provided, wherein the first decentral identifier is associated with the second decentral identifier and/or vice versa according to the a relationship representation.

5. The apparatus of claim 1, wherein at least one second decentral identifier is provided, wherein the at least one second decentral identifier is associated with the second physical entity, wherein the first decentral identifier associated with the first physical entity is connected to the second decentral identifier associated with the second physical entity, wherein the second physical entity is used to produce the first physical entity or the first physical entity is produced by using the second physical entity.

6. The apparatus of claim 1, wherein at least one second decentral identifier is provided, wherein the at least one second decentral identifier is associated with at least one second physical entity of the product supply chain and/or the recycling product chain, wherein a concatenation is generated based on a relationship representation according to which the first decentral identifier is associated with the at least one second decentral identifier.

7. The apparatus of claim 1, wherein multiple second decentral identifier(s) are provided, wherein multiple second decentral identifiers are related according to a relationship representation.

8. The apparatus of claim 1, wherein the first decentral identifier is associated with multiple physical entities of the product supply chain and/or the recycling product chain.

9. The apparatus of claim 1, wherein at least one decentral identifier is provided per physical entity of the product supply chain and/or a recycling product chain, wherein the first and second decentral identifiers are connected according to a relationship representation representing the relationship of the physical entities of the product supply chain and/or the recycling product chain.

10. The apparatus of claim 1, wherein the product passport includes a concatenation associated with multiple decentral identifiers based on a relationship representation representing the relationship of the physical entities in the product supply chain and/or a recycling product chain.

11. The apparatus of claim 1, wherein one or more hash value(s) are generated based on a relationship representation.

12. The apparatus of claim 1, wherein the product passport includes one or more second decentral identifier(s) and/or the concatenation.

13. A computer-implemented method for generating a product passport, the method comprising the steps:
receiving a request to provide a first decentral identifier associated with at least one first physical entity of a product supply chain and/or a recycling product chain;
in response to the request, providing the first decentral identifier associated with the at least one first physical entity of the product supply chain and/or the recycling product chain;
generating a product passport including the first decentral identifier and data related to the at least one first physical entity of the product supply chain and/or the recycling product chain; and
providing the product passport for access by a data consuming service controlled by a data providing service associated with a data owner.

14. A computer-implemented method for producing a product associated with a product passport, wherein the product comprises at least one first physical entity of a product supply chain and/or a recycling product chain, the method comprising the steps:
collecting product data associated with at least one first physical entity of the product supply chain and/or the recycling product chain, wherein the at least one first physical entity comprises at least one physical identifier;
assigning the physical identifier to a first decentral identifier for generating the product passport associated with the at least one first physical entity; and
generating the product passport by receiving a request to provide at least the first decentral identifier associated with product data of the at least one first physical entity and in response to the request, generating the product passport including the first decentral identifier and data related to the product data of the at least one first physical entity.

15. A computer-implemented method for using a product passport, the method comprising the steps:
receiving a request to access and/or process the product passport associated with a first decentral identifier as generated according to the method of claim 13;
optionally authenticating the product passport and/or the request to access and/or process the product passport;
based on the product passport and optionally the authentication, providing access to product data associated with at least one decentral identifier of the product passport.

16. A computer element with instructions, which when executed on one or more computing node(s) is configured to carry out the steps of the method of claim 13.

17. A product associated with a product passport, wherein the product passport including the first decentral identifier and data related to the product data is generated for the product according to the method of claim 13.

18. A computer-implemented method for using a product passport, the method comprising the steps:
receiving a request to access and/or process the product passport associated with a first decentral identifier as generated by the apparatus of claim 1;
optionally authenticating the product passport and/or the request to access and/or process the product passport;
based on the product passport and optionally the authentication, providing access to product data associated with at least one decentral identifier of the product passport.

19. A computer element with instructions, which when executed on one or more computing node(s) is configured to carry out the steps performed by the apparatus of claim 1.

20. A product associated with a product passport, wherein the product passport including the first decentral identifier and data related to the product data is generated by the apparatus of claim 1.
